(19) United States
(12) Patent Application Publication
(10) Pub. No.: US 2026/0248718 A1
(43) Pub. Date: Aug. 27, 2026

(54) PHARMACEUTICAL COMPOSITIONS OF ROFLUMILAST FOR OPHTHALMIC DELIVERY

(71) Applicant: Iolyx Therapeutics, Inc., Burlingame, CA (US)

(72) Inventors: Richard GRAHAM, Irvine, CA (US); Rozemarijn VERHOEVEN, Siler City, NC (US); Hovhannes J. GUKASYAN, Orange, CA (US); Elizabeth W. JEFFORDS, Hillsborough, CA (US); Shannon DAHL, Palo Alto, CA (US)

(73) Assignee: Iolyx Therapeutics, Inc., Burlingame, CA (US)

(21) Appl. No.: 19/547,201

(22) Filed: Feb. 23, 2026

Related U.S. Application Data
(60) Provisional application No. 63/762,264, filed on Feb. 24, 2025.

(51) Int. Cl. A61K 9/00; A61K 9/10; A61K 31/44; A61K 47/02; A61K 47/12; A61K 47/26; A61K 47/38

(52) U.S. Cl. CPC A61K 9/0051; A61K 9/10; A61K 31/44; A61K 47/02; A61K 47/12; A61K 47/26; A61K 47/38

(57) ABSTRACT

Ophthalmic pharmaceutical formulations of the phosphodiesterase-4 inhibitor, roflumilast formulated with optimized viscosity and are suitable for intravitreal or other forms of ocular administration. The compositions can improve drug administration and absorption, distribution, metabolism, and excretion (ADME) properties. The ophthalmic pharmaceutical formulations can be administered via intravitreal injection resulting in the formation of a depot in the vitreous. The depot can remain in the vitreous for a prolonged period of time resulting in therapeutic drug levels over this period of time and improved durability.

### CROSS REFERENCE TO RELATED APPLICATION

The present application claims priority to U.S. Provisional Application No. 63/762,264, filed Feb. 24, 2025, the disclosure of which is incorporated by reference herein in its entirety.

### FIELD OF THE INVENTION

The present invention relates to pharmaceutically effective ophthalmic compositions having a phosphodiesterase-4 inhibitor. The ophthalmic compositions have an optimized viscosity for back-of-eye, retinal, posterior segment, anterior segment, intraocular, orbital and peri-orbital drug delivery and durable ocular tissue exposure. Additionally, the present invention includes methods of treatment by administering said compositions for the treatment of ocular disease.

### BACKGROUND OF THE INVENTION

Roflumilast is a potent and selective long-acting inhibitor of phosphodiesterase (PDE) type 4, with anti-inflammatory and potential antineoplastic activities. Roflumilast is known to be suitable as a bronchial therapeutic agent as well as for the treatment of inflammatory disorders. Compositions containing phosphodiesterase-4 inhibitors (e.g., roflumilast, apremilast, crisaborole, dovramilast, drotaverine, ensifentrine, lotamilast, and nerandomilast) are used in human and veterinary medicine and have been proposed for the treatment and prophylaxis of diseases including but not limited to: inflammatory and allergen-induced airway disorders (e.g., bronchitis, asthma, COPD), dermatoses (e.g., proliferative, inflammatory, and allergen induced skin disorders), generalized inflammations in the gastrointestinal region (Crohn's disease and ulcerative colitis), pulmonary fibrosis, and gastrointestinal and genitourinary smooth muscle spasms. Oral pharmaceutical compositions of roflumilast are currently marketed under the tradenames Daliresp® (in the United States) and Daxas® (in Europe), and topical compositions of roflumilast cream for dermatological use are currently marked under the tradename Zoryve™ (in the United States).

Roflumilast and its synthesis are described in U.S. Pat. No. 5,712,298. It has been recognized that pharmaceutical compounds having phosphodiesterase (PDE)-4 inhibiting properties, such as roflumilast, are therapeutically effective and useful for treating inflammatory disorders, such as psoriasis and atopic dermatitis. While the therapeutic effectiveness of oral and dermal pharmaceutical compositions have been studied, there is a need for efficient, druggable, ophthalmic pharmaceutical compositions of roflumilast suitable for treating inflammatory and immune-mediated, neurodegenerative, or blood/retina barrier disorders of the eye, particularly for delivery to the anterior and posterior compartments of the eye, or into the tissues or chambers surrounding the eye and orbit. The majority of the market for anti-inflammatory and neuroprotective ophthalmic drugs today is based around antibiotics, immunosuppressants and steroids, many of which either do not meet the clinical needs of long-term inflammatory disease, or which present significant long-term comorbidity and safety issues; or the use of vascular targeted agents such as anti-VEGF's which provide a decrease in exudation or leakage, but may not effectively treat the earlier stages of disease, and need to be dosed frequently and invasively through injections every month or two months. As such, there is a high unmet need for a long-lasting anti-inflammatory and neuroprotective roflumilast ophthalmic formulation in a convenient and tolerable form, suitable for the ocular surface, anterior, or vitreous/posterior compartments of the eye.

The delivery of drugs locally to the eye is very difficult, as pharmaceutical ophthalmic agents must balance tolerability, sterility, safety, and efficacy. Developing a stable ophthalmic formulation, which can be made under sterile conditions while retaining physicochemical properties, staying within a tight range of pH and inactive ingredients which are tolerable to the eye, and which can be delivered in effective doses to the eye, is very difficult. Excipients used for ophthalmic delivery can potentially exhibit ocular toxicity and further exacerbate disorders and associated symptoms of the treated disorder.

Ophthalmic delivery is typically focused on the ocular surface, the anterior, suprachoroidal space, posterior/vitreous segment of the eye, the optic nerve, or posterior to the globe of the eye. Ocular surface formulations, often delivered by the patient as an eye drop, one to four times a day (or more, in the case of tapered steroids), have the additional challenge of requiring dosing consistency and yet flexibility to deliver effective dose despite common operator errors found in home-based patient delivery: sterility issues, variance in delivery volume, patient compliance, and accuracy in placement. Patients with long-term ocular disease also have increased sensitivity to active and inactive ingredients and preservatives, creating additional formulation challenges. Internal or posterior/vitreo retinal formulations applied by injection or other long-acting delivery vehicle are even more difficult to create, as the method of delivery (often an injection) is more invasive, requiring a much less frequent delivery cadence, with current treatments delivered by injection to the eye lasting one, two, or three months, while retaining some stability of tissue residence. The delivery of a pharmaceutical preparation to the interior or peri-orbital space is also limited by the use of fine-gauge needles, often injected into viscous environments such as vitreous humor or orbital adipose. The injection into viscous environments limits the viscosity with which it is possible or easy to insert a pharmaceutical preparation, with easy to inject formulations being preferred by physicians for the avoidance of undue pressure applied to the eye, and related safety or dosing accuracy concerns. When accessing the interior compartments of the eye via injection, regardless of the site of injection, it is desired to limit the number of applications (injections) to minimize risk to the patient of introducing infectious agents, and to limit physical stress to the sites of injection and frequency of office visits, particularly when treating chronic conditions. Patient tolerance for frequent injections may be related to severity of disease, with more severe disease states leading patients to tolerate more frequent dosing, and treatment of earlier stages of disease progression requiring less frequent dosing via internal or posterior/vitreo retinal routes.

There exists a need for efficient, highly durable drug payload within a small injection volume, for use in many ophthalmic disorders, to limit the frequency of injections. Injections are also limited to physician administration, causing a burden for both patient and physician office, and shortened durability of drug, once administered, causes repeated visits and monitoring, exacerbating the problem.

### SUMMARY OF THE INVENTION

The present invention relates to ophthalmic pharmaceutical compositions of the phosphodiesterase-4 inhibitor, roflumilast. The inventors of the subject application have determined that high viscosity formulations of drugs containing roflumilast can improve drug administration and absorption, distribution, metabolism, and excretion (ADME) properties when used in the anterior, posterior, or peri-orbital segments of the eye. In certain embodiments, the ophthalmic pharmaceutical formulations can be administered via intravitreal injection resulting in the formation of a depot in the vitreous. The depot can remain in the vitreous for a prolonged period of time resulting in therapeutic drug levels over this period of time and improved durability. This form of delivery of drug is in contrast to what is referred herein as a “snow globe effect” or “pluming.” These effects refer to the scattering of the drug formulation following delivery to produce what looks like snow or a plume-like pattern in the “snow globe” of the vitreous. These effects are undesirable and can impact the subject's field of vision and performance of both the lens and the retina if it coats the interior structures of the posterior segment of the eye. The methods disclosed herein can offer an improvement over existing ophthalmic treatments, which may require increased dosages or repeated injections due to inefficient drug properties once administered.

In certain embodiments, the ophthalmic pharmaceutical formulation comprises about 2% to about 5% w/v of roflumilast, a viscosity agent, a tonicity agent, a buffer agent, a surfactant, and water. The ophthalmic pharmaceutical composition is suitable for intravitreal or other injection-based administration into targeted tissues in the eye, or into the tissues or chambers surrounding the eye or orbit.

In certain embodiments, the ophthalmic pharmaceutical formulation has a viscosity between about 2 and about 100 centipoise (cPs). In preferred embodiments, the ophthalmic pharmaceutical formulation has a viscosity between about 2 and about 50 centipoise (cPs). In another preferred embodiment, the ophthalmic pharmaceutical formulation has a viscosity between about 2 and about 20 centipoise (cPs). In another preferred embodiment the ophthalmic pharmaceutical formulation has a viscosity between about 2 and about 20 centipoise (cPs). In another preferred embodiment the ophthalmic pharmaceutical formulation has a viscosity of about 2 and 15 centipoise (cPs). In another preferred embodiment, the ophthalmic pharmaceutical formulation has a viscosity between about 5 about 15 centipoise (cPs). In yet another preferred embodiment, the ophthalmic pharmaceutical formulation has a viscosity between about 7 and about 15 centipoise (cPs). In some embodiments, the composition can have a viscosity of at least 2 centipoise (cPs). In some embodiments, the composition can have a viscosity of at least 3 centipoise (cPs). In some embodiments, the composition can have a viscosity of at least 4 centipoise (cPs). In another embodiment, the composition can have a viscosity of at least 5, 6, or 7 centipoise (cPs).

In some embodiments, the composition can produce a sustained release of roflumilast for greater than 1 month, 2 months, 3 months, 4 months, 5 months, 6 months, 7 months, 8 months, 9 months or longer. The viscosity of the drug product can be important for the behavior and handling of injectability, which can be measured in newtons of force needed to expunge the product into a high-viscosity vitreous or other tissue.

The improved durability of the drug relative to existing therapies such as anti-angiogenic (e.g., ranibizumab, aflibercept, bevacizumab) or steroid products without a significant implantable delivery device can offer substantial advantages. For example, existing anti-angiogenic or complement inhibitor based intravitreal drug products require frequent administration (every month or two), which is less convenient for patients and suffers from increased safety risks due to the number of injections, each of which introduces the chance of exogenous infectious agents or other traumatic damage to the eye. Due to these limitations, existing therapies are often administered outside of labeled dosing on a less frequent basis, for example on an “as needed” or pro re nata (PRN) basis to try to minimize these downsides. However, using an “as needed” or suboptimal dosing frequency with these existing therapies produces a “seesaw” like effect on the thickness of the retina or control of angiogenic fluid in the eye, wherein the retinal thickness continually decreases and increases over time or hemorrhagic damage to the eye collects over time. Likewise, the retina and macula can experience reductions and increases of retinal fluid with these agents, going from dry to exudative repeatedly, which can cause additional stress on visual structures. Steroids used for inflammatory control of the posterior or vitreo-retinal segment of the eye are often provided with a longer duration, but with the safety cost of being permeated into various excipients or devices which hold the steroids in place. The difficulty of these vehicles and devices is that they are often made of chemical entities which can cause eye irritation, such as PEG, PLGA, surfactants or other foreign implant materials which continue to be invasive to the eye. In addition, the longer the exposure to steroids for inflammatory control in the back of the eye exposes the eye to extreme risk of cataracts, glaucoma, non-responsive glaucoma, retinal thinning and more. ((Hadayer A et al., Expert Opin Drug Delivery, 13 (8): 1083-91 (2016)); Lee H et al., BMC Ophthalmol 19 (1) (2019); Corpus K et al., Philipp J Ophthalmol, 40:52-56 (2015)). Indeed, in some studies, the incidence of cataracts from intravitreal triamcinolone has been reported at 81% (Islam M S et al., Eye, 21:321-23 (2007)) and 74-82% for fluocinolone acetonide intravitreal implant (Revised FDA label for Iluvien® 2014; EyePoint Pharmaceuticals Press Release re: Yutiq®, May 1, 2019). Even when the device is designed to be biodegradable after being implanted, it has been reported that over ⅔ of patients develop cataracts. (Revised FDA label for Ozurdex® June 2014). Other steroids not encased in an implant can cause snow globing or plumage with resultant visual field deficit. As a result, the therapies disclosed herein can offer an advantage as they require less frequent administration but sustained drug release without the safety risks of frequent injections or long-term exposure to damaging steroids, minimizing the downsides of existing therapies.

In certain embodiments, the ophthalmic pharmaceutical composition comprises a viscosity agent selected from the group consisting of hydroxypropyl methylcellulose, polyvinylpyrrolidone, or sodium carboxymethyl cellulose. In certain embodiments, the sodium carboxymethyl cellulose is a 7L or 7M grade. In certain embodiments, the tonicity agent comprises one or more of sodium chloride and potassium chloride. In certain embodiments, the ophthalmic pharmaceutical composition further comprises a buffer agent, preferably acetate and citrate buffers (e.g., sodium acetate and sodium citrate). In certain embodiments, the ophthalmic pharmaceutical composition further comprises a surfactant, preferably a polysorbate (e.g., polysorbate 20). In certain embodiments, the pH of the composition is between 5.5 to 7.5.

In some embodiments, an ophthalmic pharmaceutical composition comprising roflumilast can be formulated for higher viscosity. In some embodiments, a composition can be formulated with high-viscosity excipients for improved properties upon injection. In certain embodiments, an injection of an ophthalmic pharmaceutical composition comprising roflumilast can exhibit the self-aggregating depot effect. In certain embodiments, a formulation with a higher viscosity demonstrates superior drug depot effect compared to a formulation with a lower viscosity.

In certain embodiments, the ophthalmic pharmaceutical composition comprises about 0.1% to about 5% w/v of roflumilast, about 0.2% to about 1.0% w/v of sodium carboxymethyl cellulose, about 0.2% to about 0.8% w/v of sodium chloride, about 0.02% to about 0.25% w/v of polysorbate 20, about 0.005% to about 0.20% w/v of potassium chloride, about 0.005% to about 0.20% w/v of calcium chloride, about 0.005% to about 0.20% w/v of magnesium chloride, about 0.005% to about 0.20% w/v of sodium acetate, about 0.005% to about 0.20% w/v of sodium citrate, and water. In certain embodiments, the ophthalmic pharmaceutical composition comprises about 1% to about 5% w/v of roflumilast. The ophthalmic pharmaceutical composition is suitable for intravitreal administration or other injection-based administration into the eye, or into the tissues or chambers surrounding the eye. In certain embodiments, the pH of the composition is between 5.5 and 7.5.

The ophthalmic pharmaceutical composition is suitable for intravitreal administration or other injection-based administration into the eye, or into the tissues or chambers surrounding the eye. In certain embodiments, the pH of the composition is between 5.5 and 7.5.

In certain embodiments, the pharmaceutical composition has a particle size distribution characterized by a d90 value of less than or equal to about 20 μm. In certain embodiments, the pharmaceutical composition has a particle size distribution characterized by a d90 value of less than or equal to about 15 μm. In certain embodiments, the pharmaceutical composition has a particle size distribution characterized by a d90 value of less than or equal to about 10 μm.

In certain embodiments, a method for treating an eye disorder in a patient is provided. The method can comprise injecting one of the ophthalmic pharmaceutical compositions of roflumilast described herein into the eye of the patient. In certain embodiments, the pharmaceutical composition is one of the compositions described herein and comprises about 0.1% to about 5% w/v of roflumilast.

In certain embodiments, the eye disorder is selected from the group consisting of anterior, posterior, pan or intermediate uveitis; or uveitis associated with HLA-B27, juvenile idiopathic arthritis, Behcet's disease, ankylosing spondylitis, Vogt-Koyanagi-Harada Syndrome (VKH), or autoimmune disease; ocular graft vs host disease, Stevens-Johnson syndrome/toxic epidermal necrolysis (TENS), diabetic retinopathy, diabetic macular edema, retinal vein occlusion, age-related macular degeneration (AMD) including dry, geographic atrophy, or exudative AMD, choroidal neovascularization, retinal vasculitis (drug related/iatrogenic, non-infectious/sterile, or idiopathic), macular telangiectasia (including type 1, 2, and 3), normotensive or elevated intraocular pressure related glaucoma, Thyroid Eye Disease or choroidal thickening associated with thyroid eye disease (TED), Coats' disease, central serous retinopathy or chorioretinopathy, sterile or infectious endophthalmitis, retinitis, choroiditis, anterior or posterior scleritis/episcleritis, endothelial keratitis (bacterial, viral, fungal, or non-infectious in nature), and other inflammatory diseases of the anterior and posterior tissues of the eye or ocular complications of other inflammatory or autoimmune diseases, inflammation associated with inherited retinal diseases, retinitis pigmentosa, Stargardt disease, Leber congenital amaurosis, Leber hereditary optic neuropathy, Usher syndrome, X-linked retinoschisis, choroideremia, zonal occult outer retinopathy, myopia, vitreomacular adhesion, retinal detachment, choroidal detachment and hemorrhage, choroidal rupture, choroidal folds, proliferative vitreoretinopathy, idiopathic ischemia, achromatopsia, retinopathy of prematurity, gyrate atrophy, central areolar choroidal dystrophy, punctate inner choroidopathy, multifocal choroiditis, choroiditis, choroidal granuloma, choroidal dystrophy, choroidal fibrosis, acute posterior multifocal placoid pigment epitheliopathy, serpiginous choroidopathy, birdshot retinochoroidopathy, multiple evanescent white dot syndrome, retinoblastoma, choroidal melanoma, retinal lymphoma, and iatrogenic posterior or vitreous chamber inflammation.

In certain embodiments, administration of the pharmaceutical composition results in a therapeutic tissue residence of roflumilast in one or more of the following tissues and cell types: ocular surface, anterior segment, iris ciliary body, sclera, retina, macula, choroid, retinal pigment epithelium, vitreous humor, aqueous humor, optic nerve, orbital fibroblasts, orbital adipose tissue/adipocytes, orbital lymphocytes, the orbital musculature including but not limited to the superior rectus muscle, inferior rector muscle, medial rectus muscle, and lateral rectus muscle. In additional embodiments, said administration is performed intravitreally (IVT), peribulbarly, or into the suprachoroidal space. In additional embodiments, administration of the pharmaceutical composition results in a therapeutically relevant concentration of the roflumilast active metabolite, roflumilast-N-oxide to permeate one or more of the aforementioned tissues and cell types. Roflumilast N-oxide is only two-fold to threefold less potent than roflumilast, with respect to PDE4 inhibition, maintains high selectivity to other PDE isoenzymes and shows no selectivity for PDE4 subtypes (Rabe K F, Br J Pharmacol 163 (1): 53-67 (2011) which is herein incorporated by reference in its entirety).

In another embodiment, said therapeutically relevant concentrations of roflumilast and/or roflumilast-N-oxide to the said tissues and cell types, the pharmaceutical composition provides a method of treating eye disease related to the structures of the eye orbital such as thyroid eye disease (TED) Graves' orbitopathy/Graves' ophthalmopathy, thyroid eye disease associated with Hashimoto's Disease.

In certain embodiments, the pharmaceutical composition delivers a therapeutic level of roflumilast for up to 240 days to one or more of the cornea, limbus, conjunctiva, eyelids, lacrimal and Meibomian glands, lens, pupil, iris, anterior sclera, ciliary body, lacrimal glands, aqueous humor, the internal or endothelial or inner layer of the cornea, lacrimal glands, lymph nodes, posterior sclera, retina, choroid, macula, fovea, optic disc, optic nerve, vitreous humor, hyaloid canal, extraocular muscles, or orbital adipose tissue. In some embodiments, the composition can produce a sustained release of roflumilast for 30, 60, 90, 120, or 240 or greater days.

In certain embodiments, a method for treating a patient suffering from thyroid eye disease is provided. The method can comprise injecting one of the ophthalmic pharmaceutical compositions of roflumilast described herein into the eye of the patient. In certain embodiments, the ophthalmic pharmaceutical composition of roflumilast is administered without any foreign device or implant into the eye of the patient. In certain embodiments, the ophthalmic pharmaceutical composition of roflumilast is administered by intravitreal injection.

In certain embodiments, a method of manufacturing a sterile ophthalmic roflumilast pharmaceutical composition is provided. The method can include adding a viscosity agent, a tonicity agent, a surfactant, a buffer, and water to produce a roflumilast composition comprising about 0.1% w/v to about 5% w/v of roflumilast. The method can further include subjecting the roflumilast composition to gamma irradiation to produce a sterile ophthalmic roflumilast composition having a viscosity of between about 2 and about 20 cPs, and more preferably, between about 10 and about 20 cPs.

### BRIEF DESCRIPTION OF THE DRAWINGS

The accompanying drawings, which are incorporated herein and form part of the disclosure, help illustrate various embodiments of the present invention and, together with the description, further serve to describe the invention to enable a person skilled in the pertinent art to make and use the embodiments disclosed herein. The error bars in the drawings are standard deviation values.

FIG. 1 shows representative photos of a self-aggregating depot formation of an ophthalmic composition comprising roflumilast administered to a right (OD) and left (OS) eye in an animal model at days 15, 60, and 120.

FIGS. 2A-2B show representative photos of the slow degradation and decrease in size of a depot of an ophthalmic roflumilast composition in a rabbit vitreous animal model. FIG. 2A shows this at days 15, 30, 60, 88 and 120, while FIG. 2B shows this over a longer time period (Day 3, Day 15, month 1, month 2, month 3, month 4, month 5, month 6, month 7, and month 8.

FIG. 3 shows a representative photo of the depot effects in vitro for six ophthalmic formulations in phosphate-buffered saline (PBS) (about 0 cPs, top row) and simulated vitreous humor (about 5-10 cPs, bottom row), representing the most challenging condition for depot formation, and which both represent artificial ex-vivo conditions several-times fold lower than typical human vitreous.

FIGS. 4A-4H are plots of tissue residence of roflumilast and roflumilast n-oxide at 15, 30, 60, 90, and 120, days for intravitreally-administered and at 15, 30, 60, and 90 days for suprachoroidally administered roflumilast in key retinal/posterior or vitreous tissues, after a single dose (50 μL) of 3% pharmaceutical composition via intravitreal injection, or 3% suprachoroidal injection, into the vitreous humor and the suprachoroidal space, respectively.

FIG. 5 is a plot of the of the ocular tolerability scores of a dose-ranging experiment including bilateral intravitreal injections of a vehicle, 2%, and 5% of ophthalmic pharmaceutical composition in Dutch Belted Rabbits, in which a self-aggregating depot of product was visible in the vitreous out to 60 days.

FIG. 6 is a plot of tissue residence of roflumilast in blood plasma of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection. Any study days not plotted signify a plasma level below LLOQ at that time.

FIG. 7 is a plot of tissue residence of roflumilast n-oxide in blood plasma of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 8 is a plot of tissue residence of roflumilast in the aqueous humor (AH) of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 9 is a plot of tissue residence of roflumilast n-oxide in the aqueous humor (AH) of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 10 is a plot of tissue residence of roflumilast in the vitreous humor (VH) supernatant fraction of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 11 is a plot of tissue residence of roflumilast n-oxide in the vitreous humor (VH) supernatant fraction of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 12 is a plot of tissue residence of roflumilast in vitreous humor (VH) concentrate fraction of a rabbit model at various timepoints over a span of 90 days after delivery of a single dose (50 μL) of roflumilast (Formulation 4 of Table 2) administered via intravitreal injection. A single dose (35 μL) of roflumilast formulation (Formulation 1 of Table 2) was administered peribulbarly, but was unquantifiable in the VH concentrate.

FIG. 13 is a plot of tissue residence of roflumilast n-oxide in vitreous humor (VH) concentrate fraction of a rabbit model at various timepoints over a span of 90 days after delivery of a single dose (50 μL) of roflumilast (Formulation 4 of Table 2) administered via intravitreal injection. A single dose (35 μL) of roflumilast formulation (Formulation 1 of Table 2) was administered peribulbarly, but was unquantifiable in the VH concentrate.

FIG. 14 is a plot of tissue residence of roflumilast in retinal tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 15 is a plot of tissue residence of roflumilast n-oxide in retinal tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 16 is a plot of tissue residence of roflumilast in retinal pigment epithelium (RPE)/choroid of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 17 is a plot of tissue residence of roflumilast n-oxide in retinal pigment epithelium (RPE)/choroid of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 18 is a plot of tissue residence of roflumilast in lateral and medial rectus muscle tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 19 is a plot of tissue residence of roflumilast n-oxide in lateral and medial rectus muscle tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 20 is a plot of tissue residence of roflumilast in superior and inferior rectus muscle tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 21 is a plot of tissue residence of roflumilast n-oxide in superior and inferior rectus muscle tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 22 is a plot of tissue residence of roflumilast in orbital adipose tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 23 is a plot of tissue residence of roflumilast n-oxide in orbital adipose tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 24 is a plot of tissue residence of roflumilast in rostral mandibular lymph node (LN) tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 25 is a plot of tissue residence of roflumilast n-oxide in rostral mandibular lymph node (LN) tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 26 is a plot of tissue residence of roflumilast in lacrimal gland tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 27 is a plot of tissue residence of roflumilast n-oxide in lacrimal gland tissue of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 28 is a plot of tissue residence of roflumilast in the iris/ciliary body of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 29 is a plot of tissue residence of roflumilast n-oxide in the iris/ciliary body of a rabbit model at various timepoints over a span of 90 days after delivery of a single dose (50 μL) of roflumilast (Formulation 4 of Table 2) administered via intravitreal injection, or a single dose (35 μL) of roflumilast formulation (Formulation 1 of Table 2) administered via peribulbar injection.

FIG. 30 is a plot of tissue residence of roflumilast in the aqueous humor of three separate rabbit model study groups at various timepoints over a span of 240 days after roflumilast administration via intravitreal injection.

FIG. 31 is a plot of tissue residence of roflumilast in VH supernatant fraction of four separate rabbit model study groups at various timepoints over a span of 240 days after roflumilast administration via intravitreal injection.

FIG. 32 is a plot of tissue residence of roflumilast in VH concentrate fraction of four separate rabbit model study groups at various timepoints over a span of 240 days after roflumilast administration via intravitreal injection.

FIG. 33 is a plot of tissue residence of roflumilast in the retina of four separate rabbit model study groups at various timepoints over a span of 240 days after roflumilast administration via intravitreal injection.

FIG. 34 is a plot of tissue residence of roflumilast in the iris/ciliary body of four separate rabbit model study groups at various timepoints over a span of 240 days after roflumilast administration via intravitreal injection.

FIG. 35 is a plot of tissue residence of roflumilast in the retinal pigment epithelium/choroid of four separate rabbit model study groups at various timepoints over a span of 240 days after roflumilast administration via intravitreal injection.

FIG. 36 is a plot of tissue residence of roflumilast in the lateral/medial rectus muscle of three separate rabbit model study groups at various timepoints over a span of 240 days after roflumilast administration via intravitreal injection.

FIG. 37 is a plot of tissue residence of roflumilast in the superior/inferior rectus muscle of three separate rabbit model study groups at various timepoints over a span of 240 days after roflumilast administration via intravitreal injection.

FIG. 38 is a plot of tissue residence of roflumilast in the orbital adipose of four separate rabbit model study groups at various timepoints over a span of 240 days after roflumilast administration via intravitreal injection.

FIG. 39 is a plot of tissue residence of roflumilast in the rostral mandibular lymph node of three separate rabbit model study groups at various timepoints over a span of 240 days after roflumilast administration via intravitreal injection.

FIG. 40 is a plot of tissue residence of roflumilast in the lacrimal gland of three separate rabbit model study groups at various timepoints over a span of 240 days after roflumilast administration via intravitreal injection.

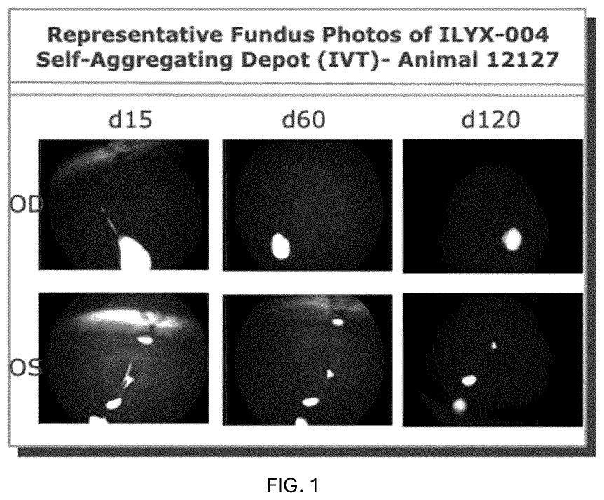

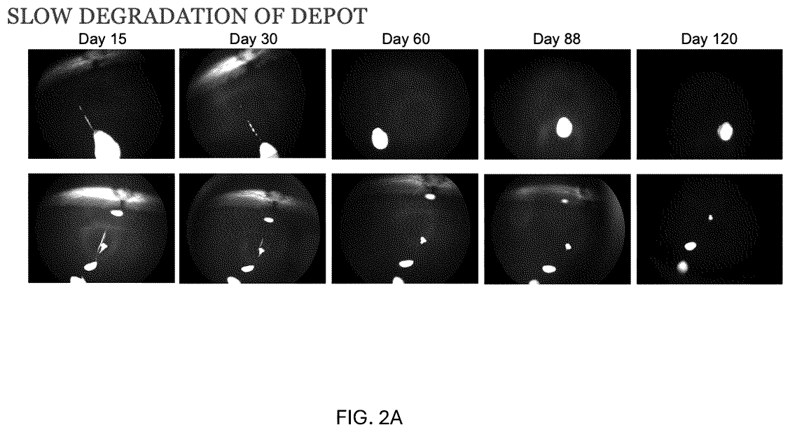

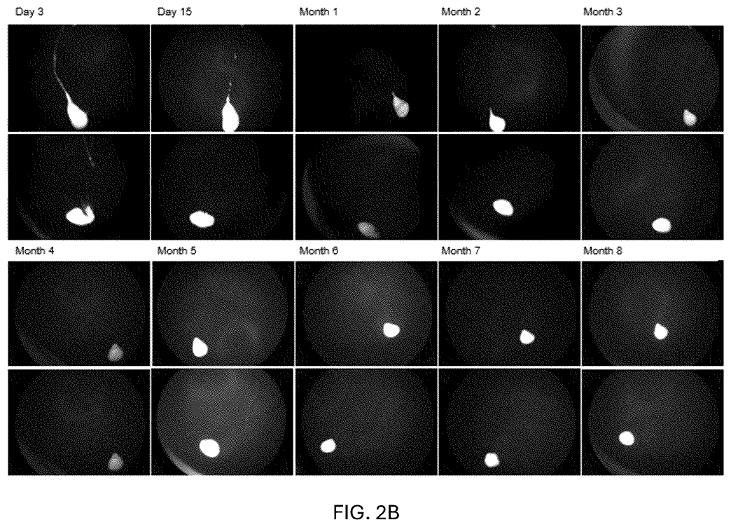

### DETAILED DESCRIPTION OF THE INVENTION

It is to be understood that the invention is not limited to the particular methodology, protocols, and reagents described herein as these may vary. It is also to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art to which this invention belongs.

All publications, patents and patent applications cited herein are hereby incorporated by reference in their entirety unless otherwise stated. Where the same term is defined in a publication, patent, or patent application and the present disclosure incorporated herein by reference, the definition in the present disclosure represents a controlling definition. For publications, patents and patent applications referenced to describe a particular type of compound, chemistry, etc., the portion relating to such compounds, chemistry, etc. is the portion of the literature incorporated herein by reference.

Note that as used herein, the singular forms “a,” “an,” and “the” include plural referents unless the context clearly dictates otherwise. Thus, for example, “active ingredient” includes a single ingredient and two or more different ingredients.

The term “about” when used in connection with a numerical value is meant to encompass numerical values within a range having a lower limit that is 5% smaller than the indicated numerical value and having an upper limit that is 5% larger than the indicated numerical value.

The term “ocular surface” refers to tissues at or near the surface of the eye, including cornea, conjunctiva, or tear film. The term “anterior chamber” or “anterior chamber ocular disease” refers to the tissues relevant to, and disease affecting the anterior or front chamber of the eye through the iris ciliary body and lens, anterior sclera, aqueous humor, and the endothelial or internal-most layer of the cornea. Anterior ocular diseases are in contrast to vitreous chamber, posterior tissues, or ocular diseases, such as those affecting the retina, and distinct from the ocular surface or those directly impacting external environment facing tissues such as the corneal conjunctival tissues, or the ocular surface and tear film. The term “vitreous chamber” or “vitreous chamber ocular disease” or “vitreal-retinal segment” refers to the tissues relevant to, or disease affecting the vitreous chamber located from the posterior of the lens, through and including retina and choroid, vitreous humor, posterior sclera, and to the optic nerve. The terms “extraorbital” or “tissues or chambers surrounding the eye” refers to tissues relevant to the external surface of the eye or the eyelid and surrounding tissues or the muscle around the eye or orbit, or the space behind the eye posterior to the globe, or to diseases related to these tissues and chambers.

The term “eye disorder,” “eye condition,” or “ocular disorder,” refer to diseases/conditions of the eye(s) that can be sight-threatening, lead to eye discomfort or disturbance, and may signal systemic health problems. The ocular surface is composed of the cornea (including particularly the corneal epithelium and stroma), limbus, conjunctiva, eyelids, lacrimal and Meibomian glands, and the interconnecting surface nerves. The anterior chamber is made up of the lens, pupil, iris, anterior sclera, ciliary body, lacrimal glands, aqueous humor, and the internal or endothelial or inner layer of the cornea, lacrimal glands and lymph nodes. The vitreous chamber is made up of the posterior sclera, retina, choroid, macula, fovea, optic disc, vitreous humor, and hyaloid canal, with the optic nerve located behind the vitreous chamber. The orbital space of the eye is made up of the eyeball and all of its internal and surface tissues, extraocular muscles (including the superior rectus, inferior rectus, medial rectus, lateral rectus, superior oblique, and inferior oblique muscles), optic nerve, blood vessels, lacrimal gland, fat/adipose pads, connective tissue, and nerves such as the oculomotor nerve, trochlear nerve, and abducens nerve. The eye as a whole is supported by the various in-eye and extra-orbital muscles and ligaments, which make up the extra-orbital space. Additionally, the eye has intra ocular muscles which include the ciliary muscle (smooth muscle that alters the shape of the lens and controls the flow of aqueous humor into Schlemm's canal), the sphincter pupillae (smooth muscle that encircles the pupil and constricts its diameter), and the dilator muscle (smooth muscle that increases the diameter of the pupil). The eyelid, also part of the eye, serves to protect, lubricate, and clean the eye. Skeletal muscles such as the levator palpebrae superioris (responsible for lifting the eyelid), orbicularis oculi (functions to close the eyelid), and frontalis muscle (assists in raising the eyebrows and slightly lifting the upper eyelid) function to control voluntary movement of the eyelid. Additionally, the superior and inferior tarsal smooth muscles (Müller's muscle) assist in lifting the upper eyelid and depressing the lower eyelid, respectively.

The term “effective” refers to an amount of a compound, agent, substance, formulation or composition that is of sufficient quantity to result in a decrease in severity of disease symptoms, an increase in frequency and duration of disease symptom-free periods, or a prevention of impairment or disability due to the disease affliction. The amount may be as a single dose or according to a multiple dose regimen, alone or in combination with other compounds, agents or substances. One of ordinary skill in the art would be able to determine such amounts based on such factors as a subject's size, the severity of a subject's symptoms, and the particular composition or route of administration selected.

“Pharmaceutically acceptable” means generally safe for administration to humans or animals. Preferably, a pharmaceutically acceptable component is one that has been approved by a regulatory agency of the Federal or a state government or listed in the U.S. Pharmacopeia, published by the United States Pharmacopeial Convention, Inc., Rockville Md., or other generally recognized pharmacopeia for use in animals, and more particularly in humans.

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

The term “roflumilast” as used in this application refers to roflumilast, its physical form, its salts, the metabolites of roflumilast including roflumilast N-oxide, and its salts unless specified otherwise or unless it is clear in context that reference is to roflumilast itself.

As used herein, the terms “subject” “or patient” most preferably refers to a human being. The terms “subject” or “patient” may include any mammal that may benefit from the compounds described herein.

A “therapeutic amount” or “therapeutically effective amount” is an amount of a therapeutic agent sufficient to achieve the intended purpose. The effective amount of a given therapeutic agent will vary with factors such as the nature of the agent, the route of administration, the size of the subject to receive the therapeutic agent, and the purpose of the administration. The effective amount in each individual case may be determined empirically by a skilled artisan according to established methods in the art.

As used herein, “treat,” “treating,” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The present invention relates to stable ophthalmic pharmaceutical compositions of the phosphodiesterase-4 inhibitor, roflumilast. Roflumilast is a compound of the formula (I):

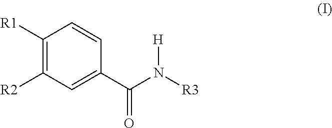

wherein R1 is difluoromethoxy, R2 is cyclopropylmethoxy and R3 is 3,5-dichloropyridin-4-yl.

Roflumilast has the chemical name N-(3,5-dichloropyridin-4-yl)-3-cyclopropylmethoxy-4-difluoromethoxybenzamide. The N-oxide of roflumilast has the chemical name 3-cyclopropylmethoxy-4-difluoromethoxy-N-(3,5-dichloropyrid-4-yl 1-oxide) benzamide. Roflumilast and its synthesis, the use of roflumilast as a phosphodiesterase (PDE) 4 inhibitor, and roflumilast formulations, were described in U.S. Pat. No. 5,712,298, which is incorporated herein by reference. The ophthalmic pharmaceutical composition can include roflumilast as a free base or a pharmaceutically acceptable salt. Exemplary salts of roflumilast are salt described in paragraphs and of U.S. Patent Application Publication No. US 2006/0084684, the disclosure of which is incorporated herein by reference. In certain embodiments, the pharmaceutical composition comprises a metabolite of roflumilast, including the N-oxide of the pyridine residue of roflumilast or salts thereof, as an active ingredient.

In certain embodiments, the ophthalmic pharmaceutical composition can comprise roflumilast in a range from about 0.1% w/v to about 6.5% w/v, or from about 0.2% w/v to about 6.5% w/v, or from about 0.3% w/v to about 6.5% w/v, or from about 1.0% w/v to about 6.5% w/v, or from about 0.3% w/v to about 5.5% w/v, or from about 0.3% w/v to about 5.5% w/v, or from about 0.5% w/v to about 5.5% w/v, or from about 0.2% w/v to about 5.0% w/v, or from about 0.2% w/v to about 5.0% w/v, or from about 1.0% to about 5.0% w/v, or from about 1.5% to about 5.0% w/v, or from about 2.0% to about 5.0% w/v. For example, the ophthalmic pharmaceutical comprises any of the following w/v percents of roflumilast: 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, etc.

In certain embodiments, the ophthalmic pharmaceutical composition can be a suspension, solution, emulsion, eye drop, eye ointment, cream, gel, a spray, an injectable formulation (intravitreal, sub-conjunctival, suprachoroidal, subtenon, periocular, peribulbar, retrobulbar, retro-orbital, or other injections), a depot formulation (whether alone or in conjunction with a depoting agent or device), an implantable absorbable polymeric device or an adsorbent contact lens. In certain embodiments, the pharmaceutical composition is intravitreal, sub conjunctival, sub retinal, intracameral, sub-tenon, periocular, peribulbar, retrobulbar, retro-orbital, suprachoroidal injection, delivery via port or drug-eluting material, cannula delivery, needle delivery, or other injection site and delivery methods.

In certain embodiments, the pharmaceutical composition may be in the form of an implant such as intravitreal implants, transscleral implants, extended release implants, biodegradable or nonbiodegradable implant materials, bioadhesive polymers, hydrogels, nanoparticles, viral vectors, adhesive spheres, micelles, microspheres, thermogels, liposomes, nanoliposomes, lipid nanoparticles, extracellular vesicles, exosomes, thermogels, mucoadhesive gels, crystals, microemulsions, nanoemulsions, emulsions, dendrimers, polyvinyl alcohol (PVA), ethylene-vinyl acetate (EVA), silicones, poly(ethylene glycol) (PEG), crosslinked poly(ethylene glycol) (PEG), poly(lactic-co-glycolic acid) (PLGA), poly-glycolic acid (PGA), and poly(caprolactones) (PCL), polyethylene terephthalate (PET), polyimide, antibodies, collagen, hyaluronic acid, extracellular matrix, silica or silicon matrix, or any combination thereof. In preferred embodiments, the pharmaceutical composition is a suspension suitable for intravitreal, suprachoroidal, or subtenon administration, wherein the active ingredient (i.e., roflumilast) is suspended in a pharmaceutical carrier and/or excipients. In certain embodiments, the roflumilast is a free-flowing re-suspendable suspension suitable for intravitreal administration or other injection.

In certain embodiments, the ophthalmic pharmaceutical composition comprises a viscosity agent, a tonicity agent, a buffer, and water. In certain embodiments, the pharmaceutical composition further comprises a surfactant. In certain embodiments, the ophthalmic pharmaceutical composition can include one or more additional excipients, including for example, a stabilizer, a preservative, a wetting agent, a diluting agent, a pH adjuster, or an absorption enhancer. In certain embodiments, the ophthalmic pharmaceutical composition can also be utilized for anterior or vitreous/posterior ophthalmic situations in the form of an injection (intravitreal, suprachoroidal, or other) as a depot, an implantable adsorbent polymeric device for any ophthalmic or surrounding tissue placement, an in situ forming gel, or a drug/device combination, wherein the active ingredient (i.e., roflumilast) is suspended with one or more of the excipients above, for example a viscosity agent, a polymer (i.e., PLGA), a surfactant, or a buffer; with or without a device or inert depot compound.

In certain embodiments, the viscosity agent is a cellulose derivative. In certain embodiments, the viscosity agent is at least one selected from the group consisting of sodium carboxymethyl cellulose, hydroxypropyl methylcellulose (HPMC), hydroxyethylcellulose (HEC), carboxymethyl cellulose (CMC), or methylcellulose. In certain embodiments, the viscosity agent is polyvinylpyrrolidone or povidone (PVP), hypromellose (HPMC), or polyvinyl alcohol (PVA). In certain embodiments, the viscosity agent is a dextran or gelatin. In certain embodiments, the viscosity agent is a carboxymethylcellulose 7L, having polymer chains that increase viscosity, but are shorter relative to other carboxymethylcellulose reagents such as carboxymethylcellulose 7M. In other embodiments, the viscosity agent is a carboxymethylcellulose 7M, having longer polymer chains than carboxymethylcellulose 7L, increasing viscosity relative to the 7L composition. In addition, the viscosity agent can include a carbomer in certain embodiments, such as a carbomer copolymer Type A or a carbomer copolymer Type B including those marketed under the trade name Carbopol® by Lubrizol®. In certain embodiments, the ophthalmic pharmaceutical formulation can comprise a viscosity agent in a range from about 0.1% w/v to about 5.0% w/v, or from about 0.1% w/v to about 4.0% w/v, or from about 0.1% w/v to about 3.0% w/v, or from about 0.1% w/v to about 2.0% w/v, or from about 0.1% to about 1.0% w/v, or from about 0.1% to about 0.8% w/v, or from about 0.2% to about 1.0% w/v, or from about 0.2% to about 0.8% w/v. For example, the ophthalmic pharmaceutical comprises any of the following w/v percents of a viscosity agent: 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, etc.

In certain embodiments, the tonicity agent is at least one selected from the group consisting of sodium chloride, potassium chloride, glycerin, and dextrose. In preferred embodiments, the tonicity agent is at least one selected from the group consisting of sodium chloride and potassium chloride. In certain embodiments, the ophthalmic pharmaceutical formulation can comprise a tonicity agent in a range from about 0.05% w/v to about 3.0% w/v, or from about 0.05% w/v to about 2.0% w/v, or from about 0.05% to about 1.0% w/v, or from about 0.1% to about 0.8% w/v, or from about 0.1% to about 0.5% w/v, or from about 0.2% to about 0.8% w/v, or from about 0.2% to about 0.5% w/v. For example, the ophthalmic pharmaceutical comprises any of the following w/v percents of a tonicity agent: 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, etc.

In certain embodiments, the surfactant is at least one selected from the group consisting of polysorbates (including, polysorbate 20, polysorbate 40, polysorbate 60, and polysorbate 80) and tyloxapol. In certain embodiments, the ophthalmic pharmaceutical formulation can comprise a surfactant in a range from about 0.02% w/v to about 3.0% w/v, or from about 0.02% w/v to about 2.5% w/v, or from about 0.02% w/v to about 2.0% w/v, or from about 0.02% to about 1.0% w/v, or from about 0.02% to about 0.5% w/v, or from about 0.02% to about 0.25% w/v. For example, the ophthalmic pharmaceutical comprises any of the following w/v percents of a surfactant: 0.02%, 0.05%, 0.075%, 0.1%, 0.15%, 0.2%, 0.25%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, etc.

In certain embodiments, the buffer is at least one selected from the group consisting of citrate, phosphate, Tris-HCl (Tris), acetate, and borate buffers. In certain embodiments, the ophthalmic pharmaceutical formulation can comprise a buffer in a range from about 0.05% w/v to about 7.5% w/v, or from about 0.05% w/v to about 5.0% w/v, or from about 0.05% to about 3.0% w/v, or from about 0.05% w/v to about 2.0% w/v, or from about 0.05% to about 1.0% w/v. For example, the ophthalmic pharmaceutical comprises any of the following w/v percents of a buffer: 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 7%, 1.8%, 1.9%, 1.0%, 2.1%, 2.2%, 2.3%, 2.4%, 2.5%, 2.6%, 2.7%, 2.8%, 2.9%, 3.0%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, etc.

In certain embodiments, the ophthalmic pharmaceutical composition can have a viscosity in a range from about 2 cPs to about 100 cPs, or from about 2 cPs to about 50 cPs, or from about 2 cPs to about 20 cPs or from about 2 cPs to about 7 cPs or from about 7 cPs to about 15 cPs, or about 7 cPs to about 10 cPs. For example, the ophthalmic pharmaceutical may have a viscosity of any of the following (in cPs): 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, etc.

In certain embodiments, the ophthalmic pharmaceutical composition can have a viscosity in a range from about 3 cPs to about 100 cPs, or from about 3 cPs to about 50 cPs, or from about 3 cPs to about 20 cPs or from about 3 cPs to about 7 cPs or from about 7 cPs to about 15 cPs, or about 7 cPs to about 10 cPs. For example, the ophthalmic pharmaceutical may have a viscosity of any of the following (in cPs): 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, etc.

The delivery of a pharmaceutical preparation to the interior or peri-orbital space is also limited by the use of fine-gauge needles, often injected into viscous environments such as vitreous humor or orbital adipose. The injection into viscous environments limits the viscosity with which it is possible or easy to insert a pharmaceutical preparation, with easy to inject formulations being preferred by physicians for the avoidance of undue pressure applied to the eye.

The ophthalmic pharmaceutical composition can be administered via a syringe needle for intravitreal administration. In certain embodiments, the ophthalmic pharmaceutical composition is easily injected from a syringe needle with minimal force. In certain embodiments, the pharmaceutical composition is capable of being injected from a 27 Gauge (G) syringe needle with a 0.41 mm outer diameter with a force of less than about 3.00 N. In certain embodiments, the pharmaceutical composition is capable of being injected from a 30 Gauge (G) syringe needle with a 0.31 mm outer diameter with a force of less than about 3.50 N. Given the ease of injection with minimal force in both 27 and 30 Gauge it is reasonable to expect that both a smaller gauge needle (30 to 33 Gauge) or a larger gauge needle (25 to 27 Gauge) would be appropriate for use. In certain embodiments, injectability was assessed by manual depression of the syringe plunger. In certain embodiments, manual administration confirmed that the pharmaceutical composition exhibits suitable injectability and can be delivered in a controlled manner without application of excessive force.

In certain embodiments, even at a concentration of up to 5% (w/v) of active pharmaceutical ingredient, the pharmaceutical composition remains injectable. In certain embodiments, the maximum injection force up to 5% (w/v) of active pharmaceutical ingredient is within a range of about 2 N to about 4 N when delivered through a syringe needle suitable for ophthalmic administration, including a 27 Gauge to 30 Gauge needle. In certain embodiments, the injection force required to inject the pharmaceutical composition of is assessed using a syringe fitted with a fine-gauge needle. In certain embodiments, about 1 ml of resuspended formulation comprising up to 5% (w/v) roflumilast was drawn into a 1 ml BD syringe fitted with either a 27 Gauge or a 30 Gauge needle, the needle being connected to a force measurement device, and the pharmaceutical composition being expelled at a controlled rate while the plunger force required to fully eject the contents was measured.

In certain embodiments, the pharmaceutical composition is capable of being injected through a 30 Gauge needle with a measured force of about 3.0 N and through a 27 Gauge needle with a measured force of about 2.9 N, indicating that the pharmaceutical composition is suitable for intravitreal injection and exhibits minimal resistance during administration.

In some embodiments, the ophthalmic pharmaceutical composition can have a viscosity sufficient to provide a sustained release of roflumilast. In some embodiments, the viscosity can be sufficient to provide a sustained release of roflumilast over a period of time, such as over about 15 days, about 30 days, about 60 days, about 90 days or about 120 days, 240 days, or about 1 month, about 2 months, about 3 months, about 4 months, about 5 months, or about 6 months. In certain embodiments, the ophthalmic pharmaceutical composition is able to provide a sustained release over these periods of time such that the drug remains at therapeutically effective levels for equal to or greater than 15 days, about 30 days, about 60 days, about 90 days or about 120 days, 240 days. In certain embodiments, the ophthalmic pharmaceutical composition provides therapeutically effective levels of roflumilast to one or more of the cornea, limbus, conjunctiva, eyelids, lacrimal and Meibomian glands, lens, pupil, iris, anterior sclera, ciliary body, lacrimal glands, aqueous humor, the internal or endothelial or inner layer of the cornea, lacrimal glands, lymph nodes, posterior sclera, retina, choroid, macula, fovea, optic disc, optic nerve, vitreous humor, hyaloid canal, or extraorbital or periorbital tissues and muscle/connective tissues.

Without being bound by theory, the properties of the composition, including the viscosity of the composition, help to control the initial injection by providing an easy to inject injection with minimal to no pluming upon injection. Once the composition has been injected into the eye of the patient and a self-aggregating depot is formed (e.g., in the vitreous of the eye outside of the patient's central field of vision), the vehicle can dissipate, and due to the properties of roflumilast, including its hydrophobic characteristics, the drug depot can persist for the duration of action.

In certain embodiments, maintenance of drug levels in vivo can be related to the drug depot effect of the injected drug product, based at least in part on the ADME of the specifically formulated drug composition, the viscosity of the composition, and the natural physicochemical properties and viscosity of the system or tissue in which the preparation is injected. In some embodiments, the viscosity of the composition can affect injection and drug properties upon delivery. Testing a low viscosity composition injection into a low viscosity system including, for example, simulated vitreous humor (VH) viscosities of between 5 and 10 cPs, can test the limits of self-aggregation and depot like behaviors. High viscosities of the composition in vivo can represent challenging conditions for some formulations of ophthalmic pharmaceutical compositions. Not being able to retain a depot in low or lower viscosity systems can also limit durability. In certain embodiments, the low viscosity compositions discussed here retain their self-aggregating depot behavior even in ex-vivo challenge conditions such as very low viscosity systems.

In some embodiments, the increased viscosity of the ophthalmic pharmaceutical composition can be achieved by varying the concentration of one or more excipients in the ophthalmic pharmaceutical composition. In some embodiments, the increased viscosity can be achieved by varying the concentration of the viscosity agent in the ophthalmic pharmaceutical composition. In some embodiments, the increased viscosity can be achieved by varying the ratio between the viscosity agent and the other excipients, for example

In certain embodiments, the ophthalmic pharmaceutical composition can be formulated such that the formulation provides an easy injection but still produces a depot following administration for a period of 30 days, 60 days, 90 days, 120 days, 240 days, or longer. In certain embodiments, the formulation can produce a prolonged therapeutic effect, which may reduce the need for frequent dosing. In certain embodiments, the depot effect can be due to increased viscosity of the formulation. In some embodiments, this property may allow the active pharmaceutical ingredient, preferably roflumilast, to be gradually released over time, maintaining therapeutic concentration within the eye while minimizing systemic exposure and potential side effects.

In certain embodiments, the ophthalmic pharmaceutical composition can be formulated to provide a reduced “snow globe” effect. The snow globe effect may be a phenomenon characterized by transient visual disturbances caused by the scattering of particles within the vitreous humor, and coating of the lens or retina with said particles. In some embodiments, the formulation can be optimized for clarity and stability. In some embodiments, the reduced snow globe effect may be due to the optimized viscosity, physiochemical properties, and excipients of the formulation and that of the system into which it is being injected. Such a formulation may, in some embodiments, provide a consistent suspension formulation that efficiently delivers the active pharmaceutical ingredient, such as a phosphodiesterase-4 inhibitor like roflumilast, and ensures patient comfort and adherence by minimizing the force used for injection, and as such, the post-administration visual and physical discomfort.

In other embodiments, the formulation may provide an opaque but consistent formulation that does not “snow globe” significantly, avoiding visual disturbances caused by particle scattering in the vitreous humor and delivers the active pharmaceutical ingredient, preferable roflumilast with patient conform and adherence by minimized post-administration visual discomfort. The ophthalmic pharmaceutical composition may be administered and deposited, forming a self-aggregating depot into the vitreous humor of the diseased or injured eye in a location within the vitreous humor so as to minimize interference with the field of vision. In certain embodiments, the ophthalmic pharmaceutical composition possesses a viscosity sufficiently low to ensure comfort, convenience, and safety during administration. This characteristic minimizes the force required for injection while enabling the formation of self-aggregating depots within the vitreal humor. The self-aggregating composition is effective in both relatively high viscosity environments, such as rabbit vitreal humor, and in the lower viscosity vitreal humor of humans, including elderly patients who may exhibit even lower viscosity levels.

In certain embodiments, the ophthalmic pharmaceutical formulation does not include any preservatives or anti-microbial agents, as most ophthalmic preservatives and anti-microbial agents are known to cause patient discomfort, burning, stinging, or irritation, and long-term exposure to these agents in a depot formulation could create a long-term irritation or inflammation.

Roflumilast can undergo hydrolysis in certain ophthalmic pharmaceutical compositions and under certain standard sterile manufacturing processes. In certain embodiments, the pH of the ophthalmic pharmaceutical compositions is adjusted to reduce the rate of hydrolysis of roflumilast.

In certain embodiments, the osmolality of the ophthalmic pharmaceutical composition is about 250 mOsm/kg to 330 mOsm/kg, and preferably, about 270 mOsm/kg to about 300 mOsm/kg, and even more preferably 270 mOsm/kg to 280 mOsm/kg.

The ophthalmic pharmaceutical compositions of the present invention are stable and exhibit a particle size distribution suitable for ophthalmic delivery. Particle size of the ophthalmic pharmaceutical composition for suspensions can be assessed using laser diffraction methods. Laser diffraction is recognized by standards and guidance agencies including ISO and ASTM and is widely used to determine particle size distributions. In conducting the assessment, the sample is passed through a laser beam, which results in laser light scattered at a range of angles. Detectors placed at fixed angles measure the intensity of light scattered at that position. A mathematical model is then applied to generate a particle size distribution.

In particle size determinations, the median value is defined as the value where half of the population resides above this point, and half resides below this point. For particle size distributions the median is called the D50. The D50 is the size that splits the distribution with half above and half below this diameter. The distribution width may also be characterized by citing one, two or three values on the x-axis, typically some combination of the D10, D50, and D90. The D50 (or the median), as discussed above, refers to the diameter wherein half of the population lies below this value. Similarly, 90 percent of the distribution lies below the D90, and 10 percent of the population lies below the D10.

In certain embodiments of the present invention, the ophthalmic pharmaceutical composition exhibits a particle size distribution characterized by a d90 value of less than or equal to about 50 μm prior to preferential processing. In certain embodiments, the ophthalmic pharmaceutical composition exhibits a particle size distribution characterized by a d90 value of from about 5 μm to about 25 μm. In certain embodiments, the pharmaceutical compositions exhibits a particle size distribution characterized by a d90 value of from about 5 μm to about 15 μm. In certain embodiments, the pharmaceutical compositions exhibit a particle size distribution characterized by a d90 value of less than or equal to 20 μm. In certain embodiments, the pharmaceutical compositions exhibit a particle size distribution characterized by a d90 value of less than or equal to 15 μm. In preferred embodiments, the pharmaceutical compositions exhibit a particle size distribution characterized by a d90 value of less than or equal to 10 μm.

In certain embodiments of the present invention, the ophthalmic pharmaceutical composition is stable and is free of impurities or has limited impurities. In certain embodiments, the pharmaceutical composition has less than about 1.0%, or less than about 0.5%, or less than about 0.2%, or essentially 0% impurities after terminal sterilization (e.g., gamma irradiation or dry heat sterilization). The amount of impurities can be assessed using an HPLC assay.

The ophthalmic pharmaceutical composition can be administered via a syringe needle for intravitreal administration. In certain embodiments, the ophthalmic pharmaceutical composition is easily injected from a syringe needle with minimal force for maximal patient comfort and precision targeting of desired injection site(s). In certain embodiments, the pharmaceutical composition is capable of being injected from a 27 Gauge (G) syringe needle with a 0.41 mm outer diameter with a force of less than about 3.00 N. In certain embodiments, the pharmaceutical composition is capable of being injected from a 30 Gauge (G) syringe needle with a 0.31 mm outer diameter with a force of less than about 3.50 N. Given the ease of injection with minimal force in both 27 and 30 Gauge it is reasonable to expect that both a smaller gauge needle (30 to 33 Gauge) or a larger gauge needle (25 to 27 Gauge) would be appropriate for use.

The inventors of the subject application have identified that roflumilast in combination with multiple viscosity agents undergo particle size growth and aggregation in certain heat transferring ophthalmic pharmaceutical manufacturing processes designed to sterilize a formulation. The inventors of the subject application have discovered certain methods of avoiding this aggregate causing heat transfer during sterile processing of the roflumilast in the same vessel as the inactive ingredients including the excipients, surfactants, etc., which can reduce the rate of particle size growth and aggregation while maintaining product potency. Mixing both sterile API with sterile inactives reduces particle aggregation by reducing the need for additional energy inputs like autoclaving, which can cause particle aggregation. In certain embodiments, slow dry heat sterilization at a temperature less than the melting point of roflumilast, gamma radiation, or other methods of sterilization of API can be used to sterilize the roflumilast while standard autoclaving can be used to sterilize the inactives before creating the final mixed formulation for an ophthalmic pharmaceutical composition which is optimized in potency, purity, and particle size, ideal for use in the eye.

In certain embodiments of the present invention, the sterility and safety of the product can be ensured via sterilization followed by sterility testing. Gamma, E-beam, or x-ray sterilization can provide terminal sterilization. With the pharmaceutical compositions listed herein, a dry heat or gamma or x-ray radiation can provide assurance of sterility, followed by sterility testing. The accuracy and extent of the gamma radiation is validated via dosimeter recordings of total radiation experienced in all quadrants of the gamma chamber. Further, sterility can be tested via standard two-week to a month screening post sterilization. The inventors have assessed both non-clinical and clinical batches in this manner, both validating adequate dry heat or terminal sterilization as well as clearing two- or four-week sterility testing with no microbial growth. Additionally, the injectable product can be controlled for endotoxins. In certain embodiments, the final injectable product can have less than 1 Endotoxin Units per milliliter (i.e., <1 EU/ml). In additional embodiments, gamma irradiation of the pharmaceutical compositions listed herein can be implemented using a standard gamma cycle of 25 to 40 kilogray (kGy) of gamma irradiation. In further embodiments, gamma irradiation can be utilized to control degradation of carboxymethylcellulose (CMC). In another embodiment, sodium CMC 7M (e.g., Formula 4 of Table 2) would be expected to increase the viscosity of the drug product from 3 cPs to 100 cPs. However, by controlling the gamma irradiation of the pharmaceutical composition (see, Choi J et al., Polymer Degradation and Stability, 93 (1): 310-15 (2008) which is incorporated herein by reference in its entirety) the viscosity increases to between 10 cPs and 15 cPs.

Terminal sterilization or dry heat can be used for a pharmaceutical composition by injection, because the composition will be directly injected into the ocular globe or surrounding tissues or chambers (whether sub-conjunctivally, intravitreally, suprachoroidally, peribulbarly, or other site). Terminal sterilization ensures that both the product and the vial are sterile. Dry heat sterilization ensures that the API added to a pre-filtered sterile excipient base will remain sterile through an aseptic process. Needles and syringes which are pre-sterilized are readily available to be used with such a pharmaceutical composition and product configuration. In certain embodiments, the product for injection could also be provided in a pre-filled syringe. In certain embodiments, the resuspendable sterile suspension in crimp-capped vial which would be used with separate, pre-sterilized needles to ensure full-process sterility. The pharmaceutical composition would be resuspended by vortexing, shaking, or mixing to resuspend the suspension, and then the product would be drawn through the pre-sterilized needle prior to injection. The pre-sterilized needle for drawing suspension can be a lower gauge needle which is then replaced with a higher gauge needle for injection (injection typically accomplished with a 27-to-30-gauge needle) or the same needle could be used for drawing and injection so long as sterile conditions are maintained.

In certain embodiments of the present invention, a method of manufacturing an ophthalmic pharmaceutical composition of roflumilast is provided. The pharmaceutical composition can include the pharmaceutical compositions described above. The method can include sterilizing the roflumilast using a form of slow dry heat or low-level radiation sterilization. The sterilization can be achieved by slow dry heat sterilization of API, excipients, or final product at a temperature less than the melting point of roflumilast (approximately 159.7° C.), terminal gamma radiation, or other methods of sterilization. The method can further include sterilizing at least one inactive ingredient selected from the group consisting of a viscosity agent, tonicity agent, surfactant, and buffer using standard autoclaving. The method can further include mixing the sterilized roflumilast with the sterilized inactive ingredient to prepare a stable ophthalmic pharmaceutical composition of roflumilast. In certain embodiments, the pharmaceutical composition that is prepared is a suspension. In certain embodiments wherein terminal gamma radiation is used, the terminal gamma radiation may impact the viscosity of the ophthalmic pharmaceutical composition of roflumilast. The terminal gamma radiation can be applied such that the final viscosity of the ophthalmic pharmaceutical composition of roflumilast is maintained within a desired range, e.g., between about 1 to about 20 cPs, between about 7 to about 15 cPs, or between about 10 to about 15 cPs.

In certain embodiments, the method can further include subjecting the stable ophthalmic pharmaceutical composition of roflumilast to clarity filtration to further mitigate particle size aggregation and to create an optimal suspension. The clarity filtration can be used to produce a stable ophthalmic pharmaceutical composition of roflumilast, wherein the pharmaceutical composition has a particle size distribution characterized by a d90 value of less than or equal to 10 μm, further differentiated for use in the eye, particularly in patients who may have sensitivity to existing ophthalmic agents, particularly those used to preserve ophthalmic products. The different formulations can react differently to the filtration process due to the differences in creation of aggregates in some formulations.

The ophthalmic pharmaceutical compositions of the present invention can be administered via intravitreal injection or injection into other sites within the eye or surrounding tissues or chambers. The pharmaceutical composition of roflumilast can be administered to the eye of a patient having an eye disorder or eye condition. In certain embodiments, the pharmaceutical compositions disclosed herein are administered as an injection to treat an eye disorder selected from the group consisting of anterior, posterior, pan or intermediate uveitis; or uveitis associated with HLA-B27, juvenile idiopathic arthritis, Behcet's disease, ankylosing spondylitis, VKH, or autoimmune disease; ocular graft vs host disease, Stevens-Johnson syndrome/TENS, diabetic retinopathy, diabetic macular edema, retinal vein occlusion, age-related macular degeneration (AMD) including dry, geographic atrophy, or exudative AMD, choroidal neovascularization, retinal vasculitis (drug related/iatrogenic, non-infectious/sterile, or idiopathic), macular telangiectasia (including type 1, 2, and 3), normotensive or elevated intraocular pressure related glaucoma Thyroid Eye Disease (TED), Graves' orbitopathy/Graves' ophthalmopathy (GO) which is an autoimmune disorder leading to hyperthyroidism, TED associated with hypothyroidism (Hashimoto's Disease), choroidal thickening associated with thyroid eye disease (TED), Coats' disease, central serous retinopathy or chorioretinopathy, sterile or infectious endophthalmitis, retinitis, choroiditis, anterior or posterior scleritis/episcleritis, endothelial keratitis (bacterial, viral, fungal, or non-infectious in nature), and other inflammatory diseases of the anterior and posterior tissues of the eye or ocular complications of other inflammatory or autoimmune diseases, inflammation associated with inherited retinal diseases, retinitis pigmentosa, Stargardt disease, Leber congenital amaurosis, Leber hereditary optic neuropathy, Usher syndrome, X-linked retinoschisis, choroideremia, zonal occult outer retinopathy, myopia, vitreomacular adhesion, retinal detachment, choroidal detachment and hemorrhage, choroidal rupture, choroidal folds, proliferative vitreoretinopathy, idiopathic ischemia, achromatopsia, retinopathy of prematurity, gyrate atrophy, central areolar choroidal dystrophy, punctate inner choroidopathy, multifocal choroiditis, choroiditis, choroidal granuloma, choroidal dystrophy, choroidal fibrosis, acute posterior multifocal placoid pigment epitheliopathy, serpiginous choroidopathy, birdshot retinochoroidopathy, multiple evanescent white dot syndrome, retinoblastoma, choroidal melanoma, retinal lymphoma, and iatrogenic posterior or vitreous chamber inflammation.

In certain embodiments, the ophthalmic pharmaceutical compositions disclosed herein are administered as an injection in the periorbital space via local, orbital, peribulbar, peri-orbital, or other injections. In certain embodiments, the ophthalmic pharmaceutical compositions disclosed herein are designed to treat eyelid or extra- or peri-orbital pain and inflammation due to trauma, autoimmune disease, microbial infection, or other systemic disease; thyroid eye disease (TED), Graves' orbitopathy/Graves' ophthalmopathy (GO) which is an autoimmune disorder leading to hyperthyroidism, TED associated with hypothyroidism (Hashimoto's Disease), meibomian gland disorders, blepharitis ocular cicatricial pemphigus, mucous membrane pemphigus, orbital inflammatory pseudotumor, idiopathic or nonspecific orbital inflammation; granulomatosis with polyangiitis (GPA), Wegener's granulomatosis, orbital (including lacrimal gland) sarcoidosis, chalazion, hordeolum, atopic dermatitis of eyelid, ocular rosacea, neuro-myelitis optica, histiocytic orbital lesions, periorbital capillary hemangiomas, or extra-orbital ocular complications of systemic sclerosis, scleroderma, or other autoimmune disease, and other diseases of the peri-orbital space.

Injections may also be used post-surgically to treat pain and inflammation of cataract, LASIK, PRK, PTK, full or partial thickness keratotomy or keratoplasty, glaucoma-related surgical procedures, inflammation related to gene or cell therapy instillation, antibody-drug conjugate associated toxicity or inflammation, side effects of Dupilumab including ocular side effects (e.g., conjunctivitis, keratitis, dry eye syndrome, blepharitis) or other iatrogenic or surgical conditions and procedures where inflammation would be a concern and injectable therapeutic intervention is appropriate. The eye disorders treatable by the methods described herein can be acute or chronic. In certain embodiments, the method is used to treat a patient who has an inflammatory or immune disorder of the eye. In certain embodiments, the inflammatory or immune disorder can be one of the above-identified disorders.

In preferred embodiments, the eye disorder is posterior, pan or intermediate uveitis; or uveitis associated with HLA-B27, juvenile idiopathic arthritis, Behcet's disease, ankylosing spondylitis, VKH, or autoimmune disease; diabetic retinopathy, diabetic macular edema, cystoid macular edema, retinal vein occlusion, age-related macular degeneration including dry, geographic atrophy, or exudative AMD and choroidal neovascularization, macular telangiectasia (including type 1, 2, and 3), normotensive or elevated intraocular pressure related glaucoma thyroid eye disease (TED), Graves' orbitopathy/Graves' ophthalmopathy (GO) or TED associated with hypothyroidism (Hashimoto's Disease), sterile or infectious endophthalmitis, glaucoma, macular telangiectasia, and vitreous or posterior chamber iatrogenic inflammation.

In certain embodiments, administration of the pharmaceutical composition results in therapeutically relevant concentrations of roflumilast in one or more of the following tissues and cell types: vitreous humor, aqueous humor, orbital fibroblasts, orbital adipose tissue/adipocytes, orbital lymphocytes, the orbital musculature including but not limited to the superior rectus muscle, inferior rector muscle, medial rectus muscle, and lateral rectus muscle. In additional embodiments, administration of the pharmaceutical composition results in therapeutic concentrations of the roflumilast active metabolite, roflumilast-N-oxide, to one or more of the aforementioned tissues and cell types. In another embodiment, the administration of the pharmaceutical composition provides a method of treating eye disease related to the structures of the eye orbital such as thyroid eye disease (TED) Graves' orbitopathy/Graves' ophthalmopathy, thyroid eye disease associated with Hashimoto's Disease.

In certain embodiments, the pharmaceutical composition is administered as a regimen, such as at regular intervals. For example, a pharmaceutical composition can be administered directly to the ocular surface as a drop or ointment once daily, twice daily, thrice daily, four times daily, once per week, twice per week, three times per week, or four times per week, monthly, as needed (PRN), or used in a treat and extend manner. In certain embodiments, the pharmaceutical composition can be administered as part of a maintenance dose or titrating dose regimen. The pharmaceutical composition can be administered for a prescribed period of time. For example, a pharmaceutical composition can be administered for a period of about two days to at least about six weeks, or until an improvement in the eye condition or disease is observed. Exemplary periods of time for the treatment regimen include one week, two weeks, one month, six weeks, two months, three months, four months, five months, six months, seven months, eight months, nine months, or one year. For example, a pharmaceutical composition can be administered as an injection or as an implantable device, depot, or adsorbable device could be administered once per week, once per month, once every 2, 3, 4, 5, 6, 7, 8, 9, 10, 11, 12, 13, 14, 15, 16 weeks, once per quarter, once every sixth months, as needed (PRN), per physician direction, or per some clinical criteria such as treat and extend or other criteria. The pharmaceutical composition can be administered as an ongoing treatment with no end.

In additional embodiments, the pharmaceutical composition is administered intravitreally (IVT) or peribulbarly (PB). In further embodiments, the pharmaceutical composition is administered by IVT or PB as at least one dose, depending on the formulation of the pharmaceutical composition and the length at which therapeutic concentrations of the pharmaceutical composition remain in the target tissue. In other embodiments, the pharmaceutical composition is administered intravitreally or peribulbarly as an induction dose followed by one or more maintenance doses of the pharmaceutical composition administered with an alternate route of administration, for example as eye drops, eye ointment, or such formulation as can be administered to the surface of the eye. In other embodiments, the pharmaceutical composition is administered after one or more maintenance doses are administered using an alternate rout of administration, for example as eye drops, eye ointment, or such formulation as can be administered to the surface of the eye. The induction dose and maintenance dose(s) of the pharmaceutical composition may also be formulated differently as any of the formulations described or contemplated herein. In another embodiment the induction dose may be of higher or equal viscosity and/or concentration than the maintenance dose. In other embodiments the induction dose may be of lower or equal viscosity and/or concentration than the maintenance dose. In some embodiments, the maintenance dose may have a formulation disclosed in U.S. patent application Ser. No. 17/948,550 (U.S. Patent Publication No. 2023/0088371A1) which is herein incorporated by reference in its entirety. In some embodiments, the induction dose may be administered and deliver a therapeutic concentration to the target tissue(s) for 1, 2, or 3 weeks or more or 1, 2, 3, 4, 5, 6, 7, 8, 9, 10, 11, or 12 months or more. In another embodiment, the maintenance dose may be administered once, twice, or three times per day, or once per week, once per month, once every 2, 3, 4, 5, 6, 7, 8, 9, 10, 11, 12, 13, 14, 15, 16 weeks, once per quarter, once every six months as needed (PRN), per physician direction or some clinical criteria as treat and extend or other criteria. The pharmaceutical composition can also be administered as an ongoing treatment with no end.

### ADDITIONAL EMBODIMENTS

The following additional numbered embodiments are provided by way of example. While various embodiments have been described herein, it should be understood that they have been presented by way of example only, and not limitation. Thus, the breadth and scope of the present disclosure should not be limited by any of the described exemplary embodiments.

Embodiment 1. An ophthalmic pharmaceutical composition comprising: (i) roflumilast; (ii) a viscosity agent; (iii) a tonicity agent; (iv) a surfactant; (v) a buffer; and (vi) water, wherein the ophthalmic pharmaceutical composition is suitable for intravitreal or other injection- or depot-based administration into the eye; and wherein the viscosity of the composition is between about 2 and about 100 cPs.

Embodiment 2. The ophthalmic pharmaceutical composition of Embodiment 1, wherein the pharmaceutical composition is capable of providing a sustained release of roflumilast over a period of about 30 days.

Embodiment 3. The ophthalmic pharmaceutical composition of Embodiment 1 or 2, wherein the pharmaceutical composition is capable of providing a sustained release of roflumilast over a period of about 60 days

Embodiment 4. The ophthalmic pharmaceutical composition of Embodiment 1, wherein the pharmaceutical composition is capable of providing a sustained release of roflumilast over a period of about 90 days.

Embodiment 5. The ophthalmic pharmaceutical composition of Embodiment 1, wherein the pharmaceutical composition is capable of providing a sustained release of roflumilast over a period of about 120 days

Embodiment 6. The ophthalmic pharmaceutical composition of Embodiment 1, wherein the composition produces a drug depot that remains in the vitreous for up to about 30 days.

Embodiment 7. The ophthalmic pharmaceutical composition of Embodiment 1, wherein the composition produces a drug depot that remains in the vitreous for up to about 60 days.

Embodiment 8. The ophthalmic pharmaceutical composition of Embodiment 1, wherein the composition produces a drug depot that remains in the vitreous for up to about 90 days.

Embodiment 9. The ophthalmic pharmaceutical composition of Embodiment 1, wherein the composition produces a drug depot that remains in the vitreous for up to about 240 days.

Embodiment 10. The ophthalmic pharmaceutical composition of Embodiment 1-9, wherein the viscosity agent is selected from a group consisting of hydroxypropyl methylcellulose, polyvinylpyrrolidone, and sodium carboxymethyl cellulose.

Embodiment 11. The ophthalmic pharmaceutical composition of Embodiment 1-10, wherein the tonicity agent is sodium chloride.

Embodiment 12. The ophthalmic pharmaceutical composition of Embodiment 1-11, wherein the buffer is sodium acetate and sodium citrate.

Embodiment 13. The ophthalmic pharmaceutical composition of Embodiment 1-12, wherein the surfactant is a polysorbate.

Embodiment 14. The ophthalmic pharmaceutical composition of Embodiment 1-13, wherein the pH of the composition is between 5.5 and 7.5.

Embodiment 15. The ophthalmic pharmaceutical composition of Embodiment 1-14, wherein the ophthalmic pharmaceutical formulation does not include any preservatives or anti-microbial agents.

Embodiment 16. The ophthalmic pharmaceutical composition of Embodiment 1-15, wherein the pharmaceutical composition has a particle size distribution characterized by a d90 value of less than or equal to about 15 μm.

Embodiment 17. The ophthalmic pharmaceutical composition of Embodiment 1-16, wherein the pharmaceutical composition has been subjected to terminal sterilization by gamma irradiation or dry heat sterilization to achieve less than about 0.2% impurities.

Embodiment 18. The ophthalmic pharmaceutical composition of Embodiment 1-17, wherein the viscosity of the composition is between about 2 and about 20 cPs.

Embodiment 19. The ophthalmic pharmaceutical composition of Embodiment 1-17, wherein the viscosity of the composition is between about 7 and about 15 cPs.

Embodiment 20. The ophthalmic pharmaceutical composition of Embodiment 1-19, wherein the pharmaceutical composition is capable of delivering therapeutically effective levels of roflumilast to one or more of the cornea, limbus, conjunctiva, eyelids, lacrimal and Meibomian glands, lens, pupil, iris, anterior sclera, ciliary body, lacrimal glands, aqueous humor, the internal or endothelial or inner layer of the cornea, lacrimal glands, lymph nodes, posterior sclera, retina, choroid, macula, fovea, optic disc, optic nerve, vitreous humor, hyaloid canal, or extraorbital or periorbital tissues and muscle/connective tissues for 240 days following administration.

Embodiment 21. The ophthalmic pharmaceutical composition of Embodiment 20, wherein the pharmaceutical composition is capable of delivering therapeutically effective levels of roflumilast to the extraorbital or periorbital tissues for 240 days following administration.

Embodiment 22. A method for treating an eye disorder in a patient comprising: injecting a high viscosity ophthalmic pharmaceutical composition of roflumilast without any foreign device or implant, into the eye of the patient, wherein the pharmaceutical composition comprises about 1.0% to about 5% w/v of roflumilast; a viscosity agent; a tonicity agent; a buffer agent; a surfactant; and water; and wherein the viscosity of the composition is between about 2 and about 100 cPs.

Embodiment 23. The method of Embodiment 22, wherein the pharmaceutical composition provides a sustained release of roflumilast in the posterior or peri-orbital space over a period of about 30 days.

Embodiment 24. The method of Embodiment 23, wherein the pharmaceutical composition remains well-tolerated throughout the duration of action.

Embodiment 25. The method of Embodiment 22, wherein the pharmaceutical composition provides a sustained release of roflumilast in the posterior or periorbital space over a period of about 60 days.

Embodiment 26. The method of Embodiment 25, wherein the pharmaceutical composition remains well-tolerated throughout the duration of action.

Embodiment 27. The method of Embodiment 22, wherein the pharmaceutical composition provides a sustained release of roflumilast in the posterior or periorbital space over a period of about 90 days.

Embodiment 28. The method of Embodiment 27, wherein the pharmaceutical composition remains well-tolerated throughout the duration of action.

Embodiment 29. The method of Embodiment 22, wherein the pharmaceutical composition provides a sustained release of roflumilast over a period of about 120 days.

Embodiment 30. The method of Embodiment 22, wherein the composition produces a drug depot that remains in the vitreous for up to about 30 days.

Embodiment 31. The method of Embodiment 22, wherein the composition produces a drug depot that remains in the vitreous for up to about 60 days.

Embodiment 32. The method of Embodiment 22, wherein the composition produces a drug depot that remains in the vitreous for up to about 90 days.

Embodiment 33. The method of Embodiment 22, wherein the composition produces a drug depot that remains in the vitreous for up to about 120 days.

Embodiment 34. The method of Embodiment 22-33, wherein the viscosity agent is selected from a group consisting of hydroxypropyl methylcellulose, polyvinylpyrrolidone, and sodium carboxymethyl cellulose.

Embodiment 35. The method of Embodiment 22-34, wherein the tonicity agent is sodium chloride.

Embodiment 36. The method of Embodiment 22-35, wherein the buffer is sodium acetate and sodium citrate.

Embodiment 37. The method of Embodiment 22-36, wherein the surfactant is a polysorbate.

Embodiment 38. The method of Embodiment 22-37, wherein the pH of the composition is between 5.5 and 7.5.

Embodiment 39. The method of Embodiment 22-38, wherein the ophthalmic pharmaceutical formulation does not include any preservatives or anti-microbial agents.

Embodiment 40. The method of Embodiment 22-39, wherein the pharmaceutical composition has a particle size distribution characterized by a d90 value of less than or equal to about 15 μm.

Embodiment 41. The method of Embodiment 22-40, wherein the pharmaceutical composition has been subjected to terminal sterilization by gamma irradiation or dry heat sterilization to achieve less than about 0.2% impurities.

Embodiment 42. The method of Embodiment 22-41, wherein the eye disorder is selected from the group consisting of anterior, posterior, pan or intermediate uveitis, uveitis associated with HLA-B27, juvenile idiopathic arthritis, Behcets disease, ankylosing spondylitis, Vogt-Koyanagi-Harada disease (VKH), or autoimmune disease, ocular graft vs host disease, Stevens-Johnson syndrome/toxic epidermal necrolysis, diabetic retinopathy, diabetic macular edema, retinal vein occlusion, age-related macular degeneration (AMD) including dry, geographic atrophy, or exudative AMD, choroidal neovascularization, retinal vasculitis (drug related/iatrogenic, non-infectious/sterile, or idiopathic), macular telangiectasia (including type 1, 2, and 3), normotensive or elevated intraocular pressure related glaucoma, thyroid eye disease (TED), choroidal thickening associated with thyroid eye disease (TED), Coats' disease, central serous retinopathy or chorioretinopathy, sterile or infectious endopthalmitis, retinitis, choroiditis, anterior or posterior sclertis/episcleritis, endothelial keratitis (bacterial, viral, fungal, or non-infectious in nature), and other inflammatory diseases of the anterior and posterior tissues of the eye or ocular complications of other inflammatory or autoimmune diseases, inflammation associated with inherited retinal diseases, retinitis pigmentosa, Stargardt disease, Leber congenital amaurosis, Leber hereditary optic neuropathy, Usher syndrome, X-linked retinoschisis, choroidemia, zonal occult outer retinopathy, myopia, vitreomacular adhesion, retinal detachment, choroidal detachment and hemorrhage, choroidal rupture, choroidal folds, proliferative vitreoretinopathy, idiopathic ischemia, achromatopsia, retinopathy of prematurity, gyrate atrophy, central areolar choroidal dystrophy, punctate inner choroidopathy, multifocal choroiditis, choroiditis, choroidal granuloma, choroidal dystrophy, choroidal fibrosis, acute posterior multifocal placoid pigment epitheliopathy, serpiginous choroidopathy, birdshot retinochoroidopathy, multiple evanescent white dot syndrome, retinoblastoma, choroidal melanoma, retinal lymphoma, or iatrogenic posterior or vitreous chamber inflammation.

Embodiment 43. The method of Embodiment 22-42, wherein the patient remains well-tolerated during the duration of action.

Embodiment 44. The method of Embodiment 22-43, wherein the injection is an intravitreal, sub conjunctival, sub retinal, intracameral, sub-tenon, periocular, peribulbar, retrobulbar, retro-orbital, or suprachoroidal injection.

Embodiment 45. The method of Embodiment 22-44, wherein the pharmaceutical composition delivers a therapeutic level of roflumilast to one or more of the cornea, limbus, conjunctiva, eyelids, lacrimal and Meibomian glands, lens, pupil, iris, anterior sclera, ciliary body, lacrimal glands, aqueous humor, the internal or endothelial or inner layer of the cornea, lacrimal glands, lymph nodes, posterior sclera, retina, choroid, macula, fovea, optic disc, optic nerve, vitreous humor, hyaloid canal, or extraorbital or periorbital tissues and muscle/connective tissues.

Embodiment 46. The method of Embodiment 45, wherein the pharmaceutical composition is capable of delivering therapeutically effective levels of roflumilast to one or more of the cornea, limbus, conjunctiva, eyelids, lacrimal and Meibomian glands, lens, pupil, iris, anterior sclera, ciliary body, lacrimal glands, aqueous humor, the internal or endothelial or inner layer of the cornea, lacrimal glands, lymph nodes, posterior sclera, retina, choroid, macula, fovea, optic disc, optic nerve, vitreous humor, hyaloid canal, or extraorbital or periorbital tissues and muscle/connective tissues for 240 days following administration.

Embodiment 47. The method of claim 46, wherein the pharmaceutical composition is capable of delivering therapeutically effective levels of roflumilast to the extraorbital or periorbital tissues for 240 days following administration.

Embodiment 48. The method of claim 22-47, wherein the viscosity of the composition is between about 2 and about 20 cPs.

Embodiment 49. The method of Embodiment 22-47, wherein the viscosity of the composition is between about 7 and about 15 cPs.

Embodiment 50. A method for treating thyroid eye disease in a patient comprising: administering an intravitreal injection of a high viscosity ophthalmic pharmaceutical composition of roflumilast without any foreign device or implant, into the eye of the patient, wherein the pharmaceutical composition comprises about 1.0% to about 5.0% w/v of roflumilast; a viscosity agent; a tonicity agent; a buffer agent; a surfactant; and water; and wherein the viscosity of the composition is between about 2 and about 100 cPs.

Embodiment 51. The method of Embodiment 50, wherein the pharmaceutical composition provides a sustained release of roflumilast in the posterior or periorbital space over a period of about 30 days.

Embodiment 52. The method of Embodiment 51, wherein the pharmaceutical composition remains well-tolerated throughout the duration of action.

Embodiment 53. The method of Embodiment 50, wherein the pharmaceutical composition provides a sustained release of roflumilast in the posterior or peri-orbital space over a period of about 60 days.

Embodiment 54. The method of Embodiment 53, wherein the pharmaceutical composition remains well-tolerated throughout the duration of action.

Embodiment 55. The method of Embodiment 50, wherein the pharmaceutical composition provides a sustained release of roflumilast in the posterior or peri-orbital space over a period of about 90 days.

Embodiment 56. The method of Embodiment 55, wherein the pharmaceutical composition remains well-tolerated throughout the duration of action.

Embodiment 57. The method of Embodiment 50, wherein the pharmaceutical composition provides a sustained release of roflumilast in the posterior or peri-orbital space over a period of about 120 days

Embodiment 58. The method of Embodiment 50, wherein the composition produces a drug depot that remains in the vitreous for up to about 30 days.

Embodiment 59. The method of Embodiment 50, wherein the composition produces a drug depot that remains in the vitreous for up to about 60 days.

Embodiment 60. The method of Embodiment 50, wherein the composition produces a drug depot that remains in the vitreous for up to about 90 days.

Embodiment 61. The method of Embodiment 50, wherein the composition produces a drug depot that remains in the vitreous for up to about 120 days.

Embodiment 62. The method of Embodiment 50-61, wherein the viscosity agent is selected from a group consisting of hydroxypropyl methylcellulose, polyvinylpyrrolidone, and sodium carboxymethyl cellulose.

Embodiment 63. The method of Embodiment 50-62, wherein the tonicity agent is sodium chloride.

Embodiment 64. The method of Embodiment 50-63, wherein the buffer is sodium acetate and sodium citrate.

Embodiment 65. The method of Embodiment 50-64, wherein the surfactant is a polysorbate.

Embodiment 66. The method of Embodiment 50-65, wherein the pH of the composition is between 5.5 and 7.5.

Embodiment 67. The method of Embodiment 50-66, wherein the ophthalmic pharmaceutical formulation does not include any preservatives or anti-microbial agents.

Embodiment 68. The method of Embodiment 50-67, wherein the pharmaceutical composition has a particle size distribution characterized by a d90 value of less than or equal to about 15 μm.

Embodiment 69. The method of Embodiment 50-68, wherein the pharmaceutical composition has been subjected to terminal sterilization by gamma irradiation or dry heat sterilization to achieve less than about 0.2% impurities.

Embodiment 70. The method of Embodiment 50-69, wherein the pharmaceutical composition delivers a therapeutic level of roflumilast to one or more of the orbital fibroblasts, orbital adipose tissue/adipocytes, orbital lymphocytes, the orbital musculature including but not limited to the superior rectus muscle, inferior rector muscle, medial rectus muscle, and lateral rectus muscle.

Embodiment 71. The method of Embodiment 50-70, wherein the viscosity of the composition is between about 2 and about 20 cPs.

Embodiment 72. The method of Embodiment 50-71, wherein the viscosity of the composition is between about 7 and about 15 cPs.

Embodiment 73. A method of manufacturing a sterile ophthalmic roflumilast pharmaceutical composition comprising: (i) adding a viscosity agent, a tonicity agent, a surfactant, a buffer, and water to produce a roflumilast composition comprising about 1.0% w/v to about 5.0% w/v of roflumilast, and (ii) subjecting the roflumilast composition to gamma irradiation to produce a sterile ophthalmic roflumilast composition having a viscosity of between about 2 and about 20 cPs.

Embodiment 74. The method of manufacture of Embodiment 73, wherein the viscosity agent is selected from a group consisting of hydroxypropyl methylcellulose, polyvinylpyrrolidone, and sodium carboxymethyl cellulose.

Embodiment 75. The method of manufacture of Embodiment 73-74, wherein the tonicity agent is sodium chloride.

Embodiment 76. The method of manufacture of Embodiment 73-75, wherein the buffer is sodium acetate and sodium citrate.

Embodiment 77. The method of manufacture of Embodiment 73-76, wherein the surfactant is a polysorbate.

Embodiment 78. The method of manufacture of Embodiment 73-77, wherein the pH of the composition is between 5.5 and 7.5.

Embodiment 79. The method of manufacture of claim 73-78, wherein the ophthalmic pharmaceutical formulation does not include any preservatives or anti-microbial agents.

Embodiment 80. The method of manufacture of claim 73-79, wherein the pharmaceutical composition has a particle size distribution characterized by a d90 value of less than or equal to about 15 μm.

Embodiment 81. The method of manufacture of Embodiment 73-80, wherein the pharmaceutical composition has less than about 0.2% impurities.

Embodiment 82. The method of manufacture of claim 73-81, wherein the viscosity of the composition is between about 7 and about 15 cP.

### EXAMPLES

The following examples illustrate certain embodiments of the invention without limitation.

While various embodiments have been described herein, it should be understood that they have been presented by way of example only, and not limitation. Thus, the breadth and scope of the present disclosure should not be limited by any of the described exemplary embodiments. Moreover, any combination of the above-described elements in all possible variations thereof is encompassed by the disclosure unless otherwise indicated herein or otherwise clearly contradicted by context.

### Example 1

An ophthalmic pharmaceutical composition having the composition set forth in Table 1 was prepared. The vehicle was prepared in a 100 mL glass bottle. Sodium carboxymethyl cellulose was weighed out and added. 90 mL of water for injection was added into the glass bottle. The resultant mixture was stirred until the sodium carboxymethyl cellulose was dissolved. The remaining excipients (except for roflumilast) were added and the mixture was stirred until the excipients were dissolved. Water was added QS to 100 mL. Roflumilast (previously jet-milled) was added to a 20 mL glass vial and 10 mL of the vehicle was added. The suspension was homogenized using a high-shear mixer (Polyton Model PT 10/35) to form a uniform suspension. The mixing time was 2 minutes at a shear rate of 8000 for a set of 30 at controlled room temperature.

TABLE 1
Ophthalmic Pharmaceutical Composition of Roflumilast

| Ingredient | % w/v |
|---|---|
| Roflumilast | 3.0% |
| Sodium carboxymethyl cellulose | 0.50% |
| Sodium chloride | 0.60% |
| Polysorbate 20 | 0.05% |
| Potassium chloride | 0.05% |
| Calcium chloride (dihydrate) | 0.05% |
| Magnesium chloride (hexahydrate) | 0.05% |
| Sodium acetate (trihydrate) | 0.10% |
| Sodium citrate (dihydrate) | 0.10% |
| 1N HCl | Adjust to pH~6.8 |
| Water for injection | q.s. ad 10.0 mL |

### Example 2

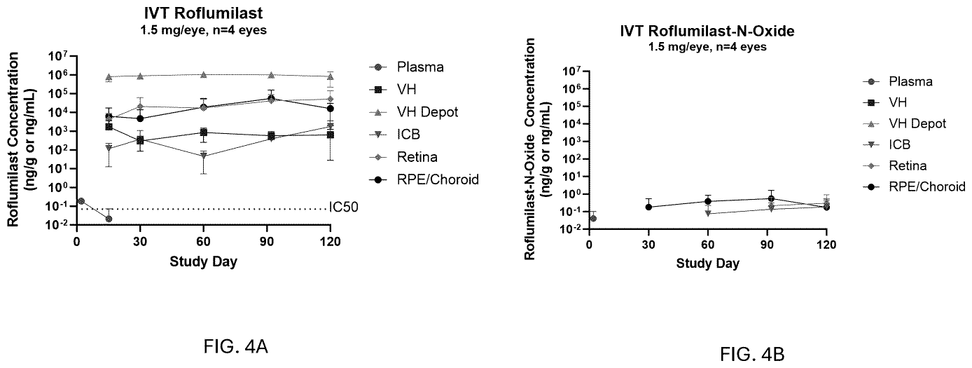

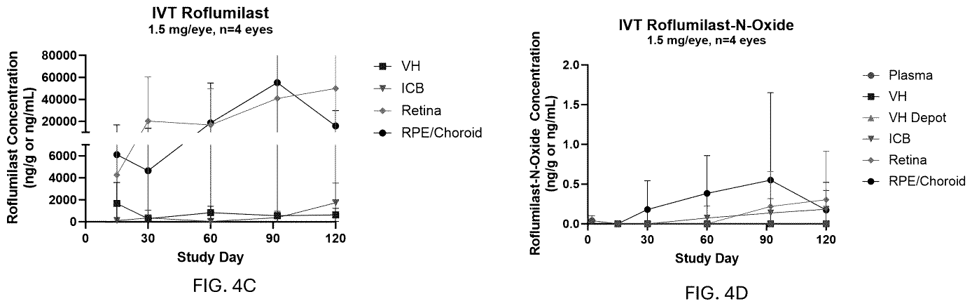

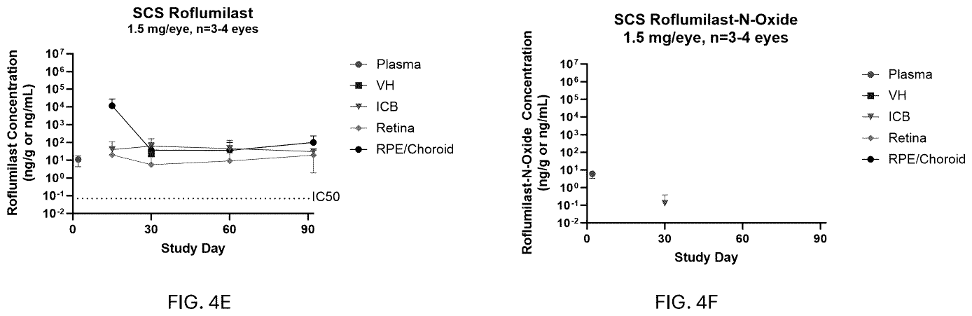

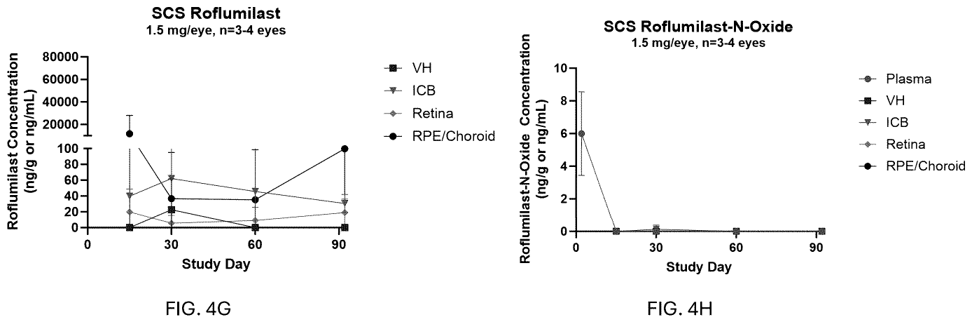

The ability to provide tissue residence at therapeutic levels was tested, and the results outlined in FIG. 4A-H. In the experiment, 10 per group Dutch Belted Rabbits were given a single dose (50 μL, 1.5 mg per eye) of the ophthalmic pharmaceutical composition described in Example 1 at day 1, either as a bilateral intravitreal (IVT) or suprachoroidal (SCS) injection. Ocular tissues, including aqueous humor, conjunctiva, sclera, cornea, iris/ciliary body (ICB), lens, vitreous humor (VH), retina, and retinal pigmented epithelium (RPE)/choroid, and plasma were collected at pre-determined time points through 4 months post-dose. Roflumilast depots (VH Depot) were collected from animals which underwent IVT administration. FIGS. 4A and 4B provide tissue residence of roflumilast and roflumilast N-oxide, respectively, following IVT injection. FIGS. 4C and 4D provide tissue residence of roflumilast on a split axis linear scale and roflumilast N-oxide on a linear scale, respectively, following IVT injection. FIGS. 4E and 4F provide tissue residence of roflumilast and roflumilast N-oxide, respectively, following SCS injection. FIGS. 4G and 4H provide tissue residence of roflumilast on a split axis linear scale and roflumilast N-oxide on a linear scale, respectively, following SCS injection. The tissue concentration results illustrate that following IVT injection, plasma exposure is minimal, unsolubilized and aggregated drug is still present in the vitreous at 30, 60, 90, and 120 days post dose, and therapeutically relevant concentrations of the pharmaceutical are present in the vitreous, iris/ciliary body, retina, and retinal pigmented epithelium (RPE)/choroid at up to 120 days post dose. Following SCS injection, plasma concentrations were higher than for IVT, and therapeutically relevant concentrations were noted in the iris/ciliary body and RPE/choroid on Day 15 through Day 90 and in the vitreous humor and on Day 30. Surprisingly, tissue concentrations from SCS injection were overall much lower than those observed with IVT injection, which may reflect the greater vascularization suprachoroidal injection region.

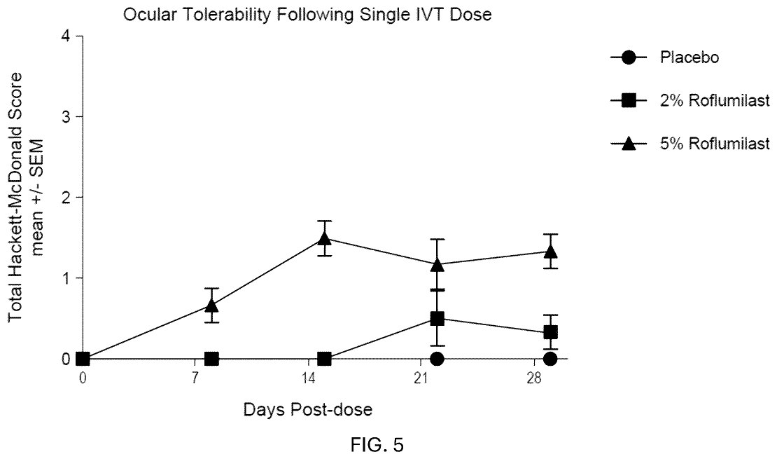

FIG. 5 shows the ocular tolerability scores for 3/group Dutch Belted Rabbits given a single bilateral intravitreal administration of the formulation described in Example 1, either a vehicle/0%, 2% or 5% ophthalmic pharmaceutical composition (Example 1 with modified concentration of roflumilast) and watched for 28 days. The ocular tolerability dose-ranging study indicates that the product is generally tolerable, with no injection site issues, or general inflammation, with no snowglobe effect in the vitreous or aggregation on the lens or retina. The product created a self-aggregating depot, with no particles noted throughout the vitreous, or aggregation on the lens, both of which are important for vision. The 5% composition resulted in a slightly higher incidence/severity of vitreal cells, but the 2% composition was found to be generally well tolerated. On days 88 and 120, both IVT and SCS administrations remained well tolerated (data not shown). Some particulates were observed, though it was unclear whether they were vitreal cells or drug particulates.

### Example 3

A set of ophthalmic pharmaceutical compositions having the compositions set forth in Table 2 was prepared. The compositions were formulated with viscosity-enhancing components and prepared in a similar manner as the composition set forth in Example 1. Composition 1 in

Table 2 is identical to the composition in Table 1.

TABLE 2
High Viscosity Ophthalmic Roflumilast Compositions
Tested in PBS and Artificial Vitreous Humor

| | Formulation# | | | | | |
|---|---|---|---|---|---|---|
| Ingredient | 1 | 2 | 3 | 4 | 5 | 6 |
| Roflumilast | 3% | 1.5% | 3% | 3% | 3% | 3% |
| Sodium | 0.50% | 0.50% | 0.50% | | 0.50% | 0.50% |
| Carboxymethyl | | | | | | |
| cellulose | | | | | | |
| (CMC)7L | | | | | | |
| Sodium | | | | 1% | | |
| Carboxymethyl | | | | | | |
| cellulose | | | | | | |
| (CMC)7M | | | | | | |
| Poloxamer 407 | | | | | 15% | |
| Poloxamer 188 | | | | | | 15% |
| Sodium | 0.60% | 0.60% | 0.60% | 0.60% | 0.60% | 0.60% |
| Chloride | | | | | | |
| Polysorbate 20 | 0.05% | 0.05% | 0.05% | 0.05% | 0.05% | 0.05% |
| Potassium | 0.05% | 0.05% | 0.05% | 0.05% | 0.05% | 0.05% |
| Chloride | | | | | | |
| Calcium | 0.05% | 0.05% | 0.05% | 0.05% | 0.05% | 0.05% |
| Chloride | | | | | | |
| (dihydrate) | | | | | | |
| Magnesium | 0.05% | 0.05% | 0.05% | 0.05% | 0.05% | 0.05% |
| Chloride | | | | | | |
| (hexahydrate) | | | | | | |
| Sodium Acetate | 0.10% | 0.10% | 0.10% | 0.10% | 0.10% | 0.10% |
| (trihydrate) | | | | | | |
| Sodium Citrate | 0.10% | 0.10% | 0.10% | 0.10% | 0.10% | 0.10% |
| (dihydrate) | | | | | | |
| 1N NaOH or | Adjust to | Adjust to | Adjust to | Adjust to | Adjust to | Adjust to |
| 1N HCl | pH ~6.8 | pH ~6.8 | pH ~6.8 | pH ~6.8 | pH ~6.8 | pH ~6.8 |
| Water for | QS | QS | QS | QS | QS | QS |
| Injection | | | | | | |

### Example 4

The ability of ophthalmic pharmaceutical compositions to maintain a drug depot in a low viscosity system including a phosphate buffered saline (PBS) (with a viscosity of about 1 cPs) and in simulated vitreous humor (with a viscosity of between about 5 and about 10 cPs) was tested. These conditions were considered a challenge for optimal depoting properties of the examples, as the human vitreous is typically ~20 to ~22,500 cPs (See Tram N K et al., Bioeng. Biotechnol. 6:199 (2018)), even with increased liquefaction of the vitreous that takes place with increased age (Id. and Sebag J et al., 1 252-62 (1987) which are herein incorporated by reference into their entireties. Seven formulations were tested, including the composition of Example 1 with the formulation described in both Table 1 and Table 2 (Formulation 1), a composition with a lower concentration of roflumilast Table 2 (Formulation 2), a composition with optimized particle size distribution (PSD) that underwent additional wet milling resulting in a D90 3 times smaller than Formulation 1, Table 2 (Formulation 3), a composition with two-fold increased sodium carboxymethyl cellulose (CMC) that is also comprised of medium chain length CMC (Formulation 4), which are longer than the CMC chains of the other compositions in Table 2, two thermogel formulations which are liquid at room temperature and become solid at body temperature (Formulations 5 and 6) and a control formulation (Kenalog mimic, data not shown).

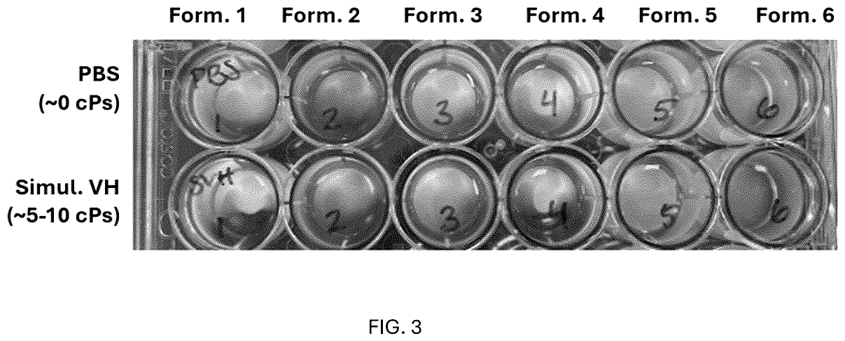

The results are set forth in FIG. 3. FIG. 3 shows twelve wells of a tissue culture plate, with the top row having 2 mL of PBS in each well and the bottom row having 2 mL simulated vitreous humor (SVH). 50 μL of each formulation presented in Table 2 were injected into the 2 mL PBS or 2 mL SVH. Column 1 of the plate indicates wells with contents injected with Formulation 1 of Table 2 (Form 1). Column 2 of the plate indicates wells with contents injected with Formulation 2 of Table 2 (Form. 2). Column 3 of the plate indicates wells with contents injected with Formulation 3 of Table 2 (Form. 3). Column 4 of the plate indicates wells with contents injected with Formulation 4 of Table 2 (Form. 4). Column 5 of the plate indicates wells with contents injected with Formulation 5 of Table 2 (Form. 5). Column 6 of the plate indicates wells with contents injected with Formulation 6 of Table 2 (Form. 6). Overall, the formulations injected well, except Formulations 5 (Form. 5) and 6 (Form. 6), which presented challenging injections. As shown in FIG. 3, Formulations 1 and 4 (columns 1 and 4) performed the best and produced a drug depot that remained in the PBS (top row) or simulated vitreous humor (bottom row) after administration despite the challenging low viscosity conditions. These results are consistent with formulations possessing a viscosity sufficient to provide a sustained release of roflumilast even in challenging conditions (such as simulated vitreous humor with cPs between about 5 and about 10).

### Example 5

The pharmacokinetic profile of modified Formulations 1 and 4 from Table 2 over 3 months following a single bilateral administration via intravitreal (IVT) or peribulbar (PB) routes respectively in Dutch Belted Rabbits was determined. The subject animals were all female and aged between 4-8 months at the start of the study.

Animals were randomized and assigned to one of two groups. The first group underwent an intravitreal surgical procedure where a needle was inserted into the eye and the test article dispensed into the vitreous humor (Formulation 4 with a concentration of roflumilast of 31.53 mg/mL in a 50 μL volume [1.6 mg/eye]). The process was repeated for the contralateral eye. The second group was administered test article peribulbarly (Formulation 1 with a concentration of roflumilast of 28.67 mg/mL in a 35 μL volume [1 mg/eye]). The process was repeated for the contralateral eye.

Ocular examinations were performed for both groups at baseline, days 3 and 15, and months 1, 2, and 3 for remaining animals. Fundoscopy was also performed on both groups at baseline, day 3, day 15 (for the Formulation 4 group) and months 1, 2, and 3 for the remaining animals. Plasma was collected on days 3, 8, 15, and months 1, 2, and 3.

Tissues were collected from animals on days 3 and 15 as well as months 1, 2, and 3. The specific tissues that were collected included the superior/inferior eyelids, lacrimal gland, extraocular muscles, orbital adipose tissue, the aqueous humor (AH), vitreous humor (VH), bulbar conjunctiva, sclera, cornea, lens, the iris ciliary body (ICB), retina, choroid/retinal pigment epithelium (RPE), and optic nerve. The vitreous humor was centrifuged and analyzed as supernatant and concentrate fractions.

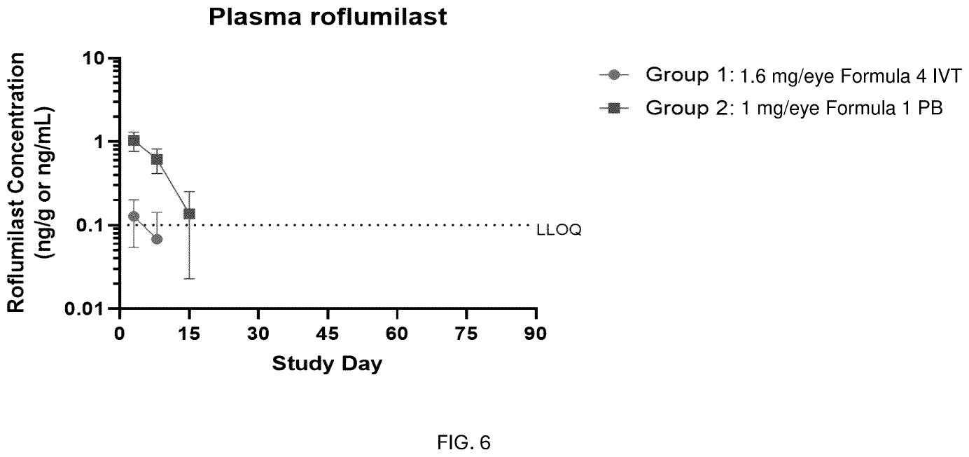

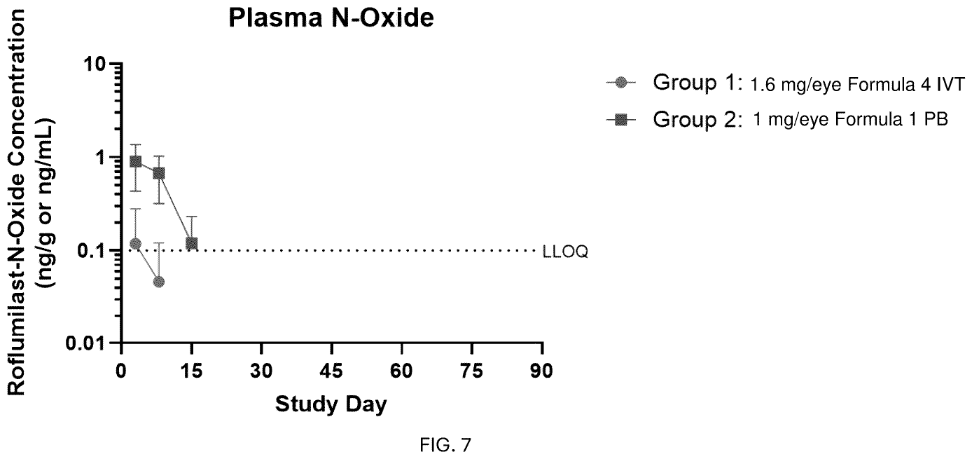

FIG. 6 shows the mean±standard error of the mean (SEM) tissue residence of roflumilast in the plasma of the subject rabbits over a span of 90 days after administration of roflumilast. The levels of roflumilast in blood plasma resulting from peribulbar injections were greater than the levels observed in blood plasma resulting from IVT administration. This was expected due to the higher vascularization in the eye orbital where peribulbar injections are made. (LLOQ denotes the lower limit of quantification). The plasma residence of roflumilast N-oxide metabolite is provided in FIG. 7 for the same timepoints. The peribulbar concentrations were quantifiable at days 3 and 8, but fell almost to the LLOQ by day 15, while the IVT concentrations were near the lower limit of quantification at day 3 and unquantifiable thereafter. This was also expected as it is understood that roflumilast undergoes gut and hepatic first pass metabolism system to form its N-oxide metabolite once exposed to the vascular system.

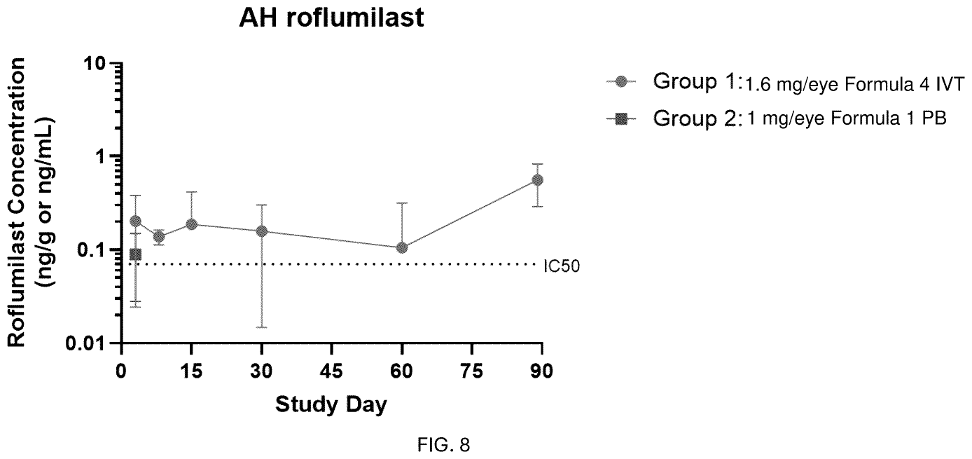

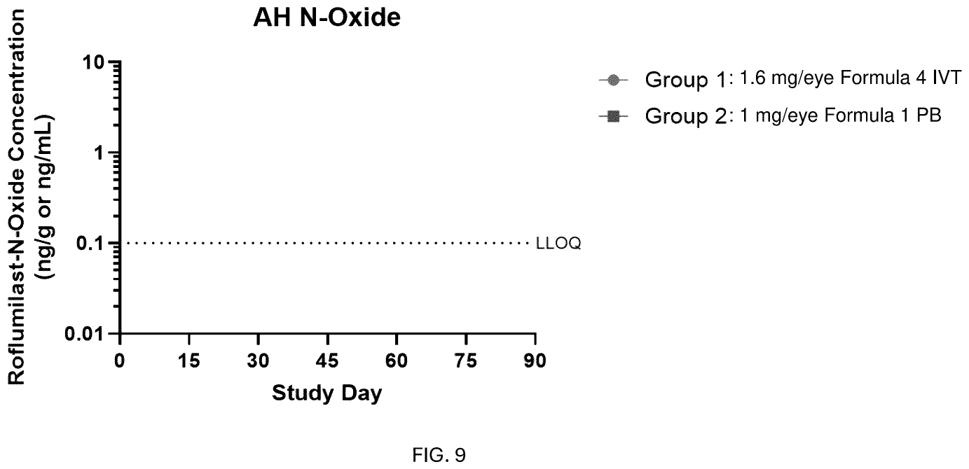

FIG. 8 shows the mean±standard error of the mean (SEM) tissue residence of roflumilast in the eye aqueous humor (AH) over a span of 90 days after administration of roflumilast in the vitreous humor. Roflumilast administered by peribulbar injection was at the lower limit of quantification on day 1, while roflumilast administered via IVT was relatively steady over three months. The lack of notable transit of roflumilast from the vitreous humor administration site to the aqueous humor was surprising and reveals how well the formulation maintains the compound near the intended site of action. FIG. 9 shows the corresponding tissue residence of roflumilast N-oxide, with no detectable levels resulting from either route of administration.

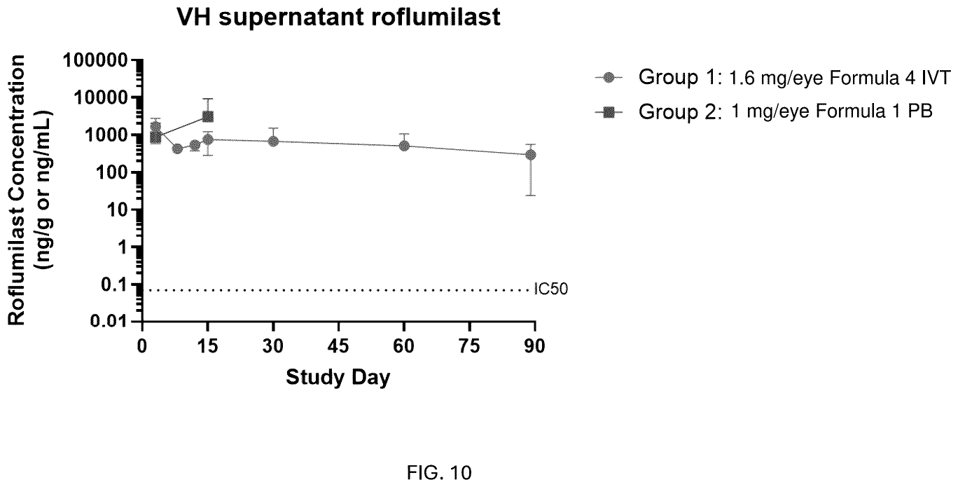

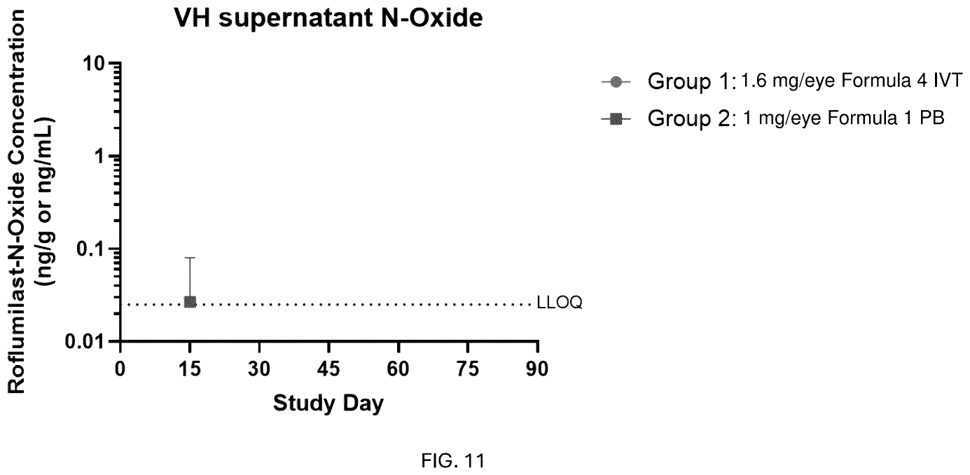

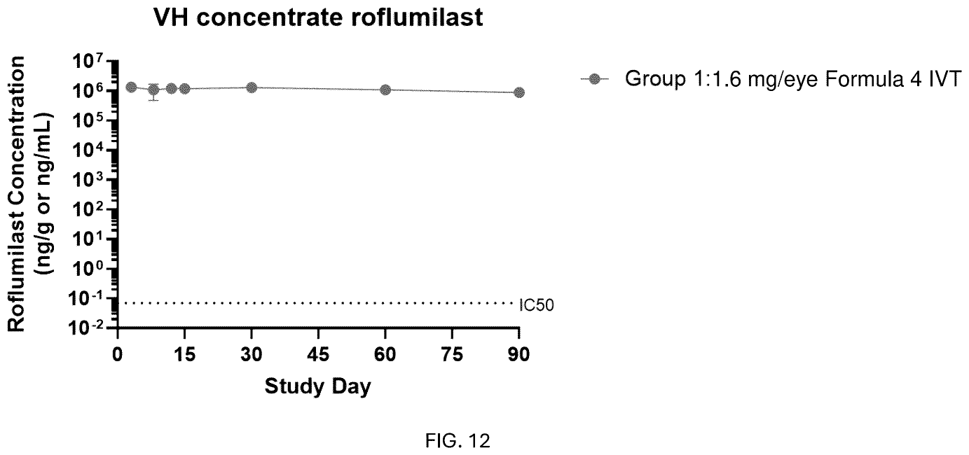

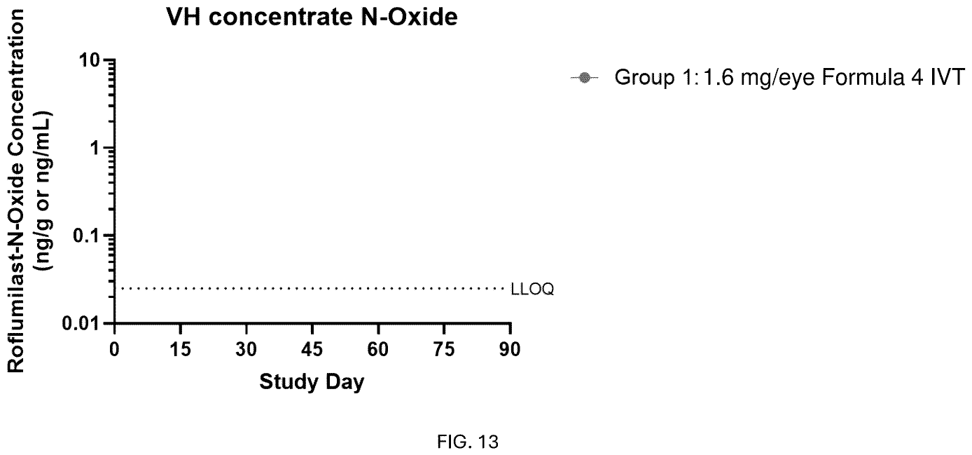

FIG. 10 shows the mean±standard error of the mean (SEM) tissue residence of roflumilast in the vitreous humor (VH) supernatant fraction of the eye over a span of 90 days after administration of roflumilast. Data points for days 1, 8, 12, 15, and 30 were plotted for the group treated via IVT. Both treatment groups after 15 days show relatively comparable levels of roflumilast in the supernatant fraction of the vitreous humor. However, for the peribulbar group, those concentrations were unquantifiable after day 15 while the IVT group maintained stable concentrations throughout the 90-day span. FIG. 11 shows the corresponding tissue residence of roflumilast N-oxide, with no detectable levels resulting from either route of administration. FIG. 12 shows the mean±standard error of the mean± (SEM) concentrations of roflumilast in the vitreous humor concentrate fraction in the group treated via IVT injection over a span of 90 days after administration of roflumilast. The concentration remained steady throughout the 3-month study. No detectable concentrations at these timepoints were observed in animals administered roflumilast via the peribulbar route. FIG. 13 shows the corresponding tissue residence of roflumilast N-oxide, with no detectable levels resulting from either route of administration.

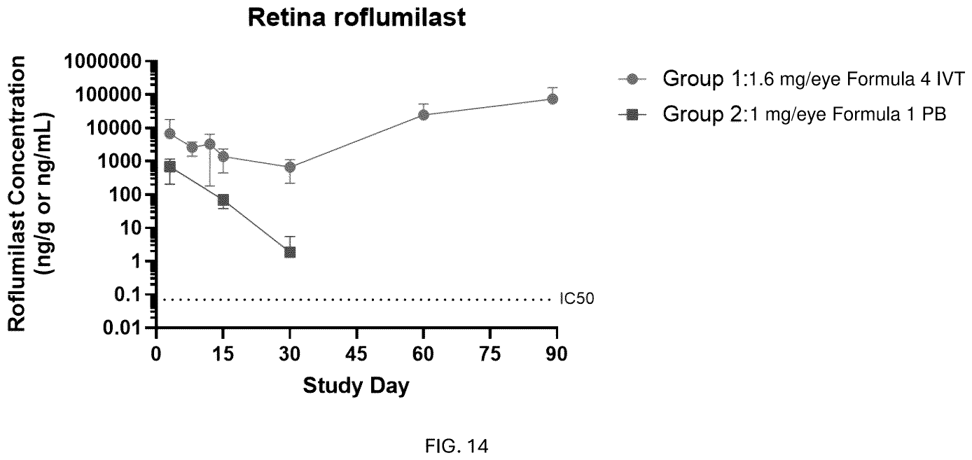

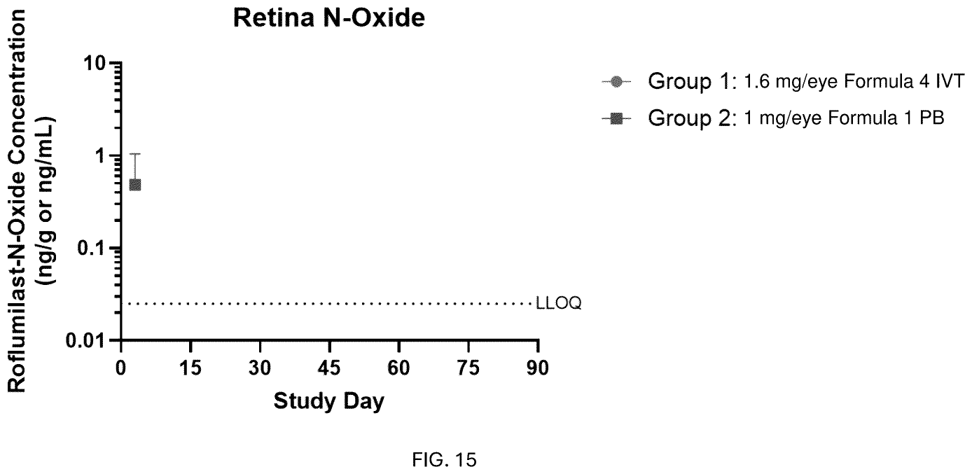

FIG. 14 shows the mean±standard error of the mean (SEM) tissue residence of roflumilast in the retina over a span of 90 days after administration of roflumilast. The group treated via IVT injection demonstrated a relatively greater concentration of roflumilast in the retina than those subjects treated via peribulbar injection, with roflumilast decreasing significantly by day 30 in the peribulbar group. In contrast, the tissue residence increased at days 60 and 90 for the IVT group. This was expected since the roflumilast administered peribulbarly is more proximal to the ophthalmic vein and carotid circulation than is the interior of the eye, resulting in increased systemic distribution. FIG. 15 shows the corresponding tissue residence of roflumilast N-oxide in the retina. Only on day 1 was there a detectable level of the N-oxide metabolite via the peribulbar route, while the IVT route produced no detectable retinal tissue distribution at any time point.

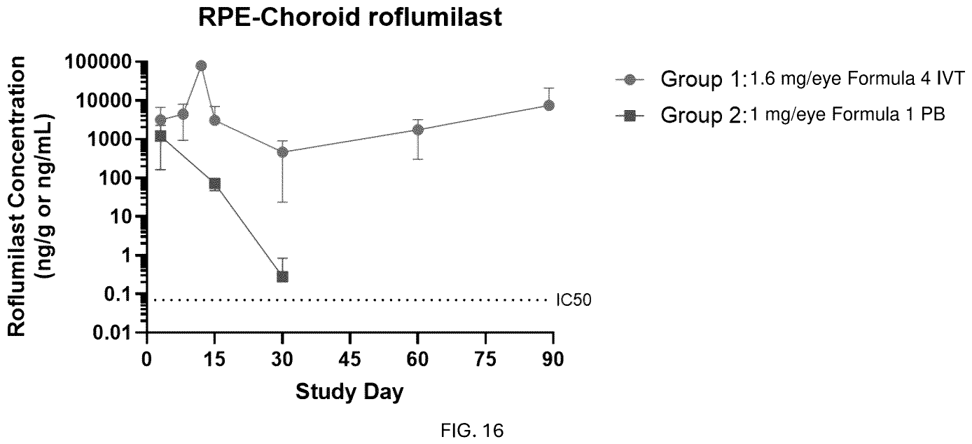

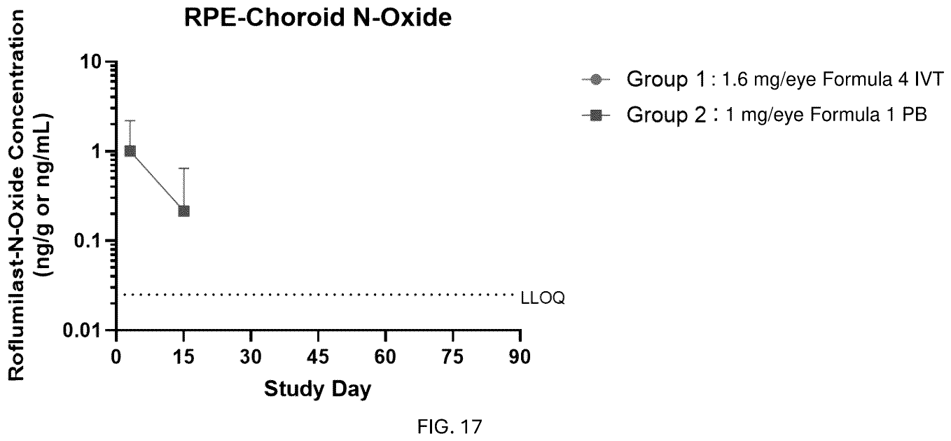

FIG. 16 shows the mean±standard error of the mean± (SEM) tissue residence of roflumilast in the choroid/retinal pigment epithelium (RPE) over a span of 90 days after administration of roflumilast. The IVT group showed relatively high concentration levels of roflumilast in the choroid/RPE on days 1, 10, 12, 15, and 30, while the group treated peribulbarly showed similar tissue residence of roflumilast on day 1, with concentrations falling over time on days 15 and 30 and becoming unquantifiable at later time points. In contrast, the tissue residence increased at days 60 and 90 for the IVT group. FIG. 17 shows the corresponding tissue residence of roflumilast N-oxide in choroid/RPE. Only the peribulbar group showed detectable levels of the N-oxide metabolite at days 1 and 15, falling to unquantifiable concentrations thereafter.

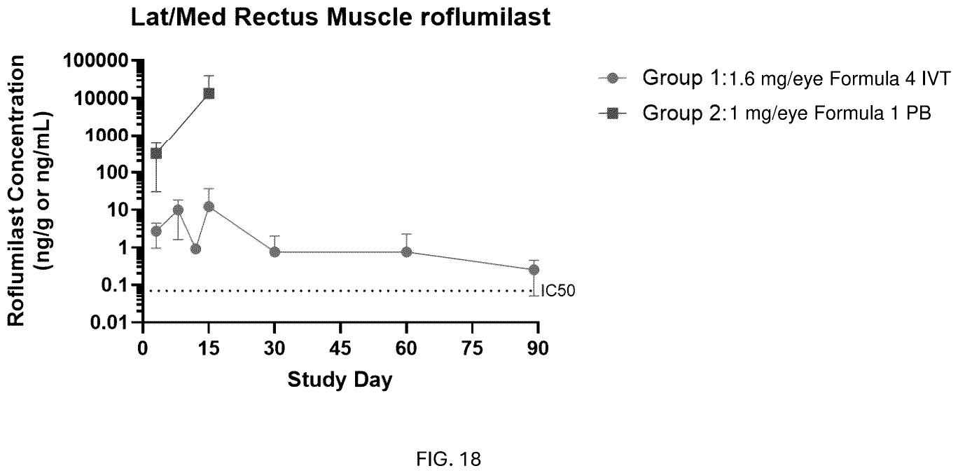

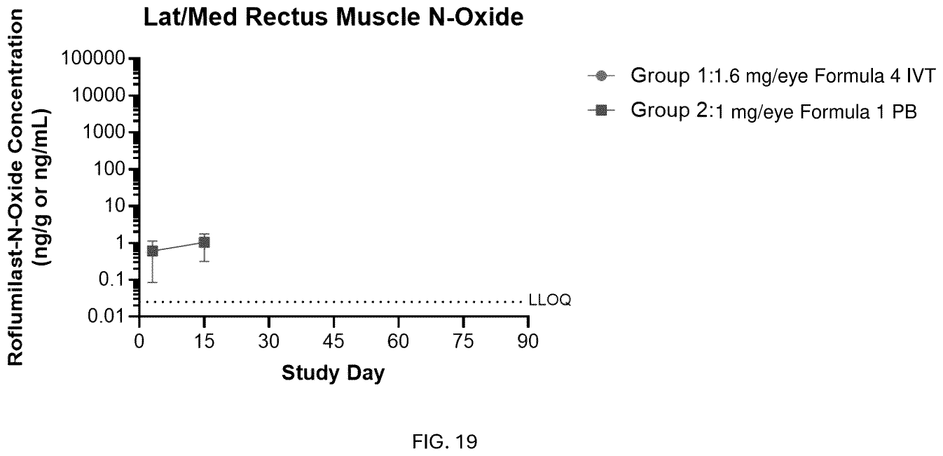

FIG. 18 shows the mean±standard error of the mean± (SEM) tissue residence of roflumilast in the lateral and medial rectus muscles over a span of 90 days after administration of roflumilast. The group treated peribulbarly exhibited greater levels of roflumilast on days 1 and 15 compared to the group treated via IVT injection. This was expected since the lateral and medial rectus muscles are in the region of the eye orbital where the peribulbar injection was administered and is better vascularized than the vitreous. Surprisingly, roflumilast concentrations were also observed in the IVT group's muscle tissues despite their reduced proximity to the site of roflumilast administration, even after 30 days, with those concentrations remaining therapeutically relevant. This suggests that the IVT route of administration can serve as an option for the treatment of eye diseases like thyroid eye disease, Graves' orbitopathy/Graves' ophthalmopathy, thyroid eye disease associated with Hashimoto's Disease. These are currently treated systemically (oral or intravenous corticosteroids or intravenous teprotumumab, a monoclonal antibody inhibitor of insulin-like growth factor-1 receptor) or locally in alternative treatments such as surgery. All are either invasive, have significant side effects, or require repeated intravenous doses over several weeks. Peribulbar concentrations fell below the level of quantification after day 15. FIG. 19 shows the corresponding tissue residence of roflumilast N-oxide in the lateral and medial rectus muscles for both groups. The peribulbar group only showed detectable levels of the N-oxide metabolite at days 1 and 15. The IVT group showed no quantifiable levels.

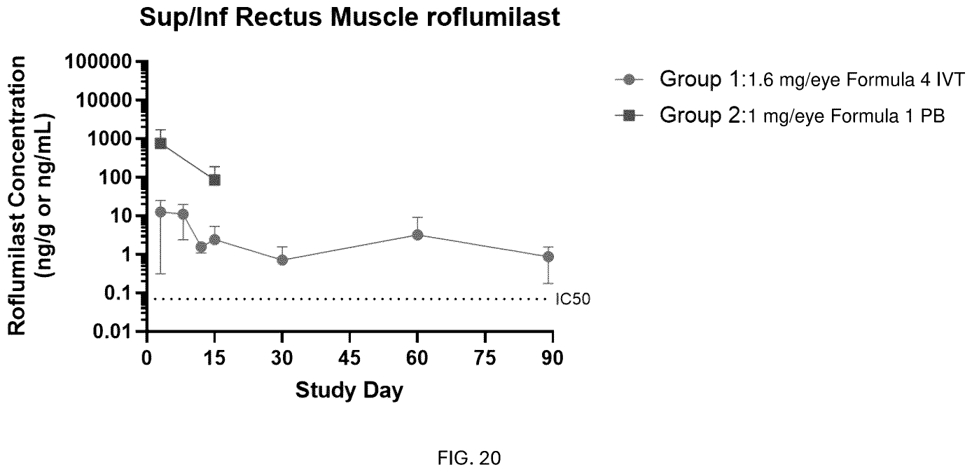

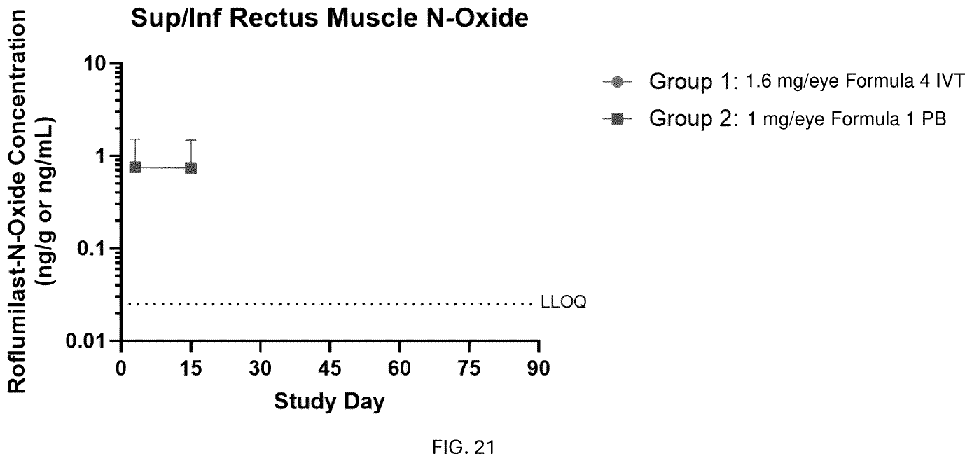

FIG. 20 shows the mean±standard error of the mean± (SEM) tissue residence of roflumilast in the superior and inferior rectus muscles over a span of 90 days after administration of roflumilast. The group treated peribulbarly exhibited greater levels of roflumilast on days 1 and 15 compared to the group treated via IVT injection. Again, this was expected since the superior and inferior rectus muscles are in the region of the eye orbital where the peribulbar injection was administered and is better vascularized than the vitreous. Surprisingly, roflumilast concentrations were also observed in the IVT group's muscle tissues despite their reduced proximity to the site of roflumilast administration throughout the 90-day span, with those concentrations being therapeutically relevant. This again suggests that the IVT route of administration can serve as an option for the treatment of eye diseases like thyroid eye disease, Graves' orbitopathy/Graves' ophthalmopathy, thyroid eye disease associated with Hashimoto's Disease. These are currently treated systemically (oral or intravenous corticosteroids or intravenous teprotumumab, a monoclonal antibody inhibitor of insulin-like growth factor-1 receptor) or locally in alternative treatments such as surgery. All are either invasive, have significant side effects, or require repeated intravenous doses over several weeks. Peribulbar concentrations did fall below the level of quantification after day 15. FIG. 21 shows the corresponding tissue residence of roflumilast N-oxide in the superior and inferior rectus muscles for both groups. The peribulbar group only showed detectable levels of the N-oxide metabolite at days 1 and 15. The IVT group showed no quantifiable levels.

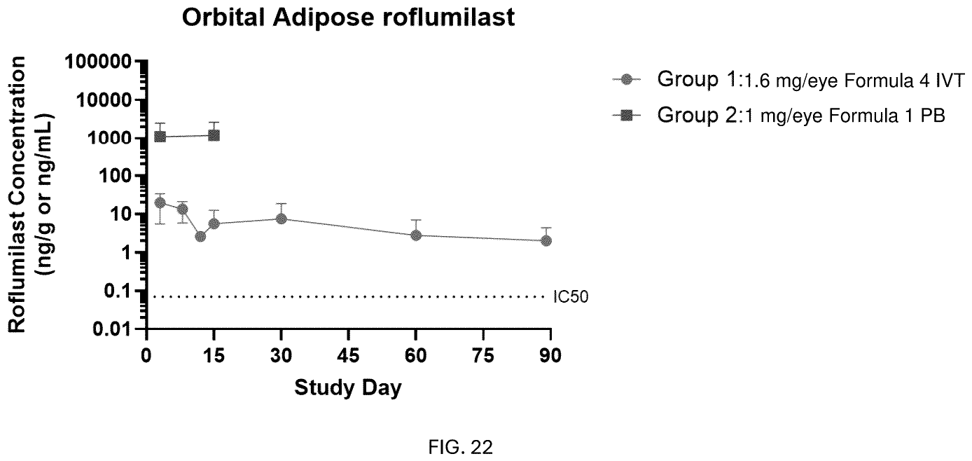

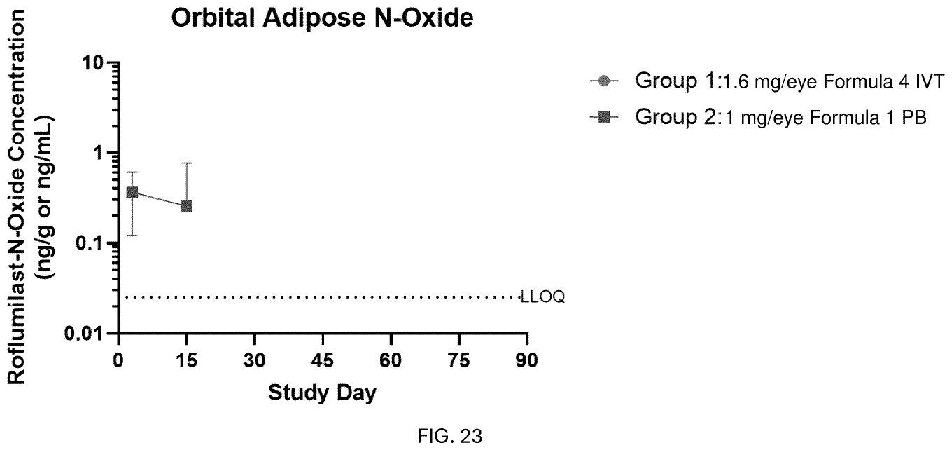

FIG. 22 shows the mean±standard error of the mean± (SEM) tissue residence of roflumilast in orbital adipose tissue over a span of 90 days after administration of roflumilast. The group treated peribulbarly exhibited higher levels of roflumilast on days 1 and 15 compared to the group treated via IVT injection. This was expected since the orbital adipose tissue is in the region of the eye orbital where the peribulbar injection was administered and is better vascularized than the vitreous. Surprisingly, roflumilast concentrations were also observed in the orbital adipose tissue despite their reduced proximity to the site of roflumilast administration throughout the 90-day span and at therapeutically relevant concentrations. This again suggests that the IVT route of administration can serve as an option for the treatment of eye diseases like thyroid eye disease, Graves' orbitopathy/Graves' ophthalmopathy, thyroid eye disease associated with Hashimoto's Disease. These are currently treated systemically (oral or intravenous corticosteroids or intravenous teprotumumab, a monoclonal antibody inhibitor of insulin-like growth factor-1 receptor) or locally in alternative treatments such as surgery. All are either invasive, have significant side effects, or require repeated intravenous doses over several weeks. In contrast, peribulbar concentrations fell below the level of quantification after day 15. FIG. 23 shows the corresponding tissue residence of roflumilast N-oxide in the orbital adipose tissue for both groups. The peribulbar group only showed detectable levels of the N-oxide metabolite at days 1 and 15. The IVT group showed no quantifiable levels.

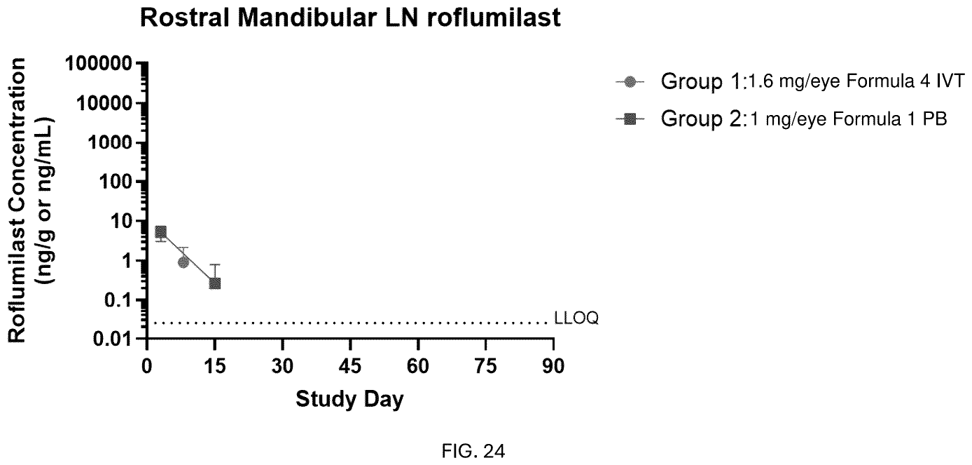

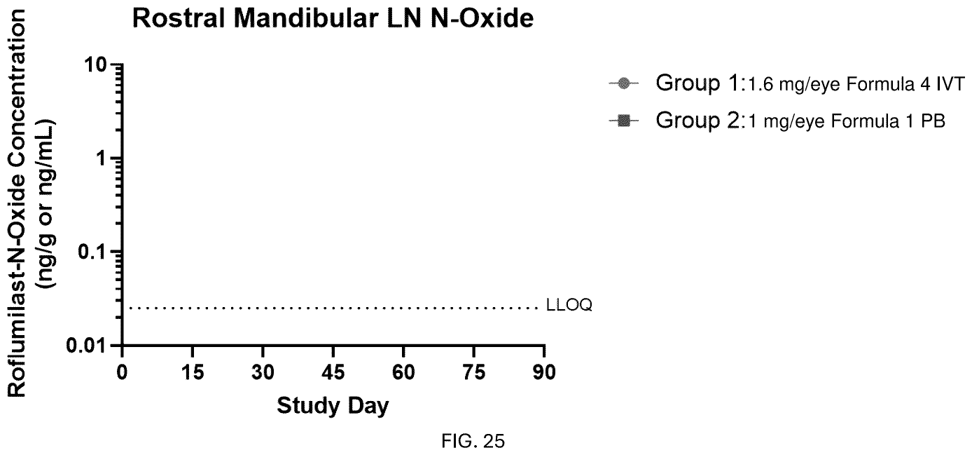

FIG. 24 shows the mean±standard error of the mean± (SEM) tissue residence of roflumilast in the rostral mandibular lymph node (LN) tissue over a span of 90 days after administration of roflumilast. Concentrations from both groups showed relatively similar levels on day 8, with detectable concentrations at day 15 only in the peribulbar group. FIG. 25 shows the corresponding tissue residence of roflumilast N-oxide in rostral mandibular lymph node (LN) tissue for both groups. Neither group produced quantifiable levels at any time.

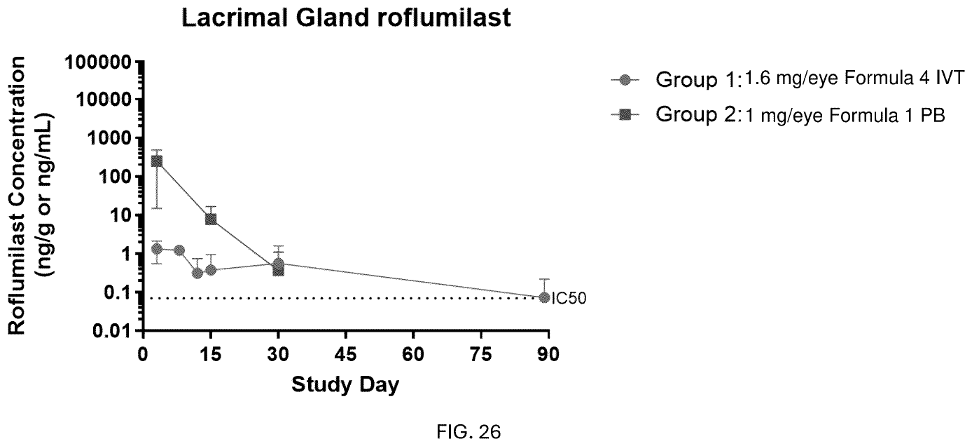

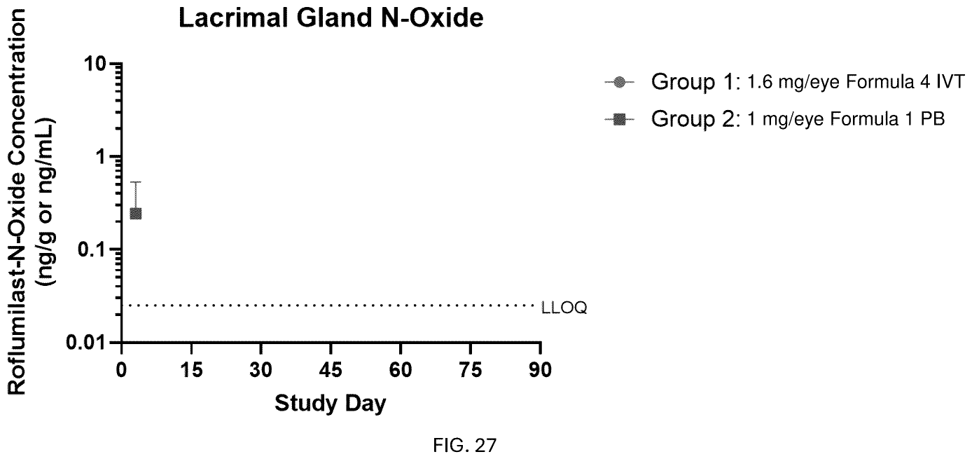

FIG. 26 shows the mean±standard error of the mean± (SEM) tissue residence of roflumilast in the lacrimal gland over a span of 90 days after administration of roflumilast. Concentrations from the peribulbar group at days 3 and 15 were greater than those of the IVT group, but the concentrations were comparable at day 30. After day 30, concentrations of the peribulbar group were undetectable and those of the IVT group were just at the quantifiable threshold. FIG. 27 shows the corresponding tissue residence of roflumilast N-oxide in the lacrimal gland for both groups. The peribulbar group only showed detectable levels of the N-oxide metabolite at day 1. The IVT group showed no quantifiable levels.

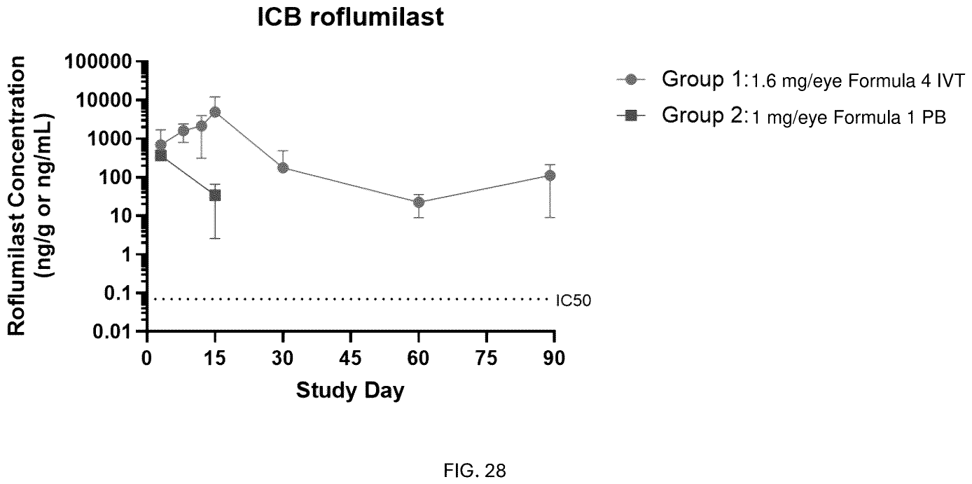

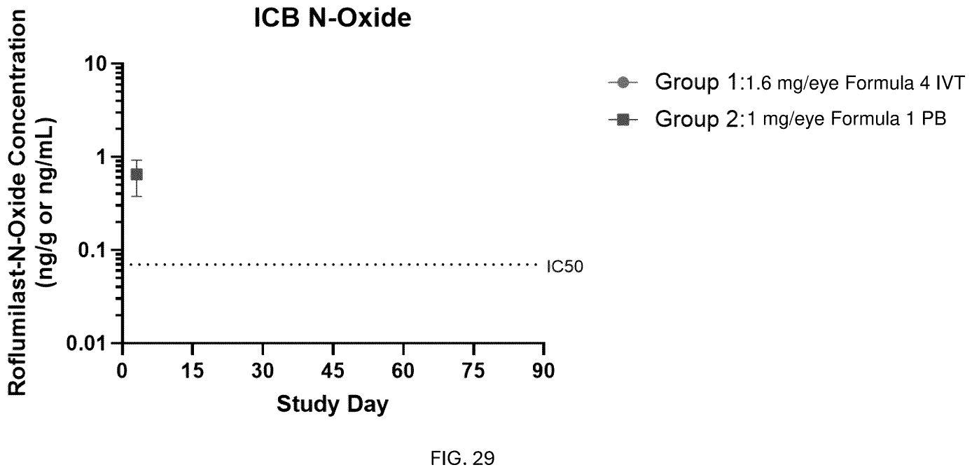

FIG. 28 shows the mean±standard error of the mean (SEM) tissue residence of roflumilast in the iris/ciliary body over a span of 90 days after administration of roflumilast. Concentrations from the peribulbar group at days 1 and 15 were at therapeutically relevant levels but became unquantifiable at later timepoints. Concentrations of the IVT group were similar to the peribulbar group at day 1 and rose over the first 15 days. At days 30, 60, and 90, those concentrations decreased but remained relatively stable at therapeutically relevant levels through the study. FIG. 29 shows the corresponding tissue residence of roflumilast N-oxide in the lacrimal gland for both groups. On day 1, the N-oxide metabolite was detectable, but both groups were unquantifiable thereafter.

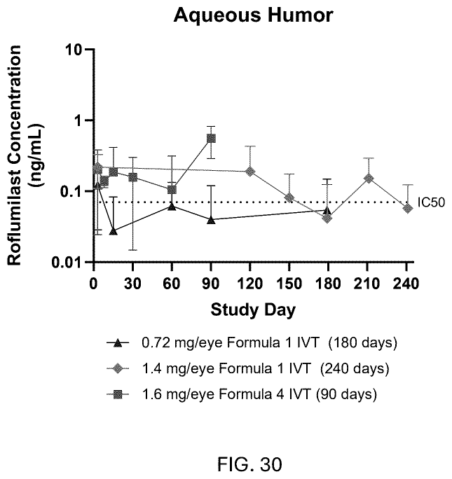

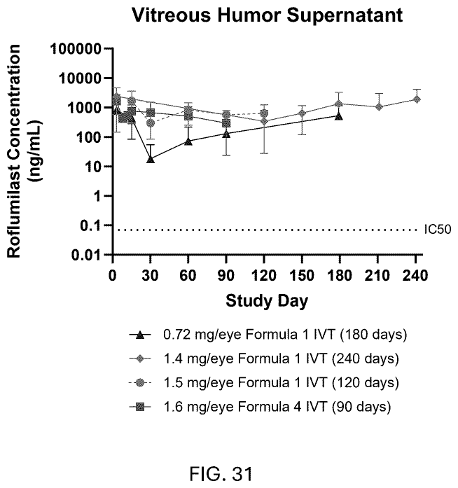

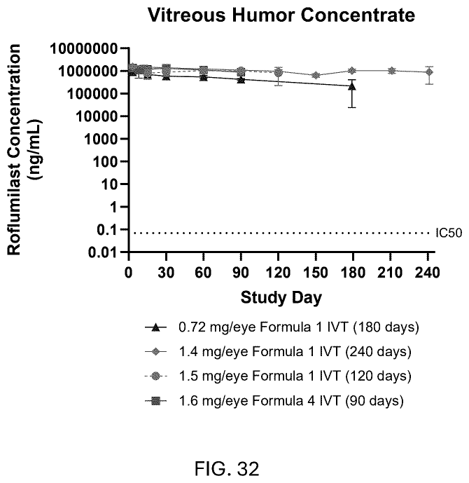

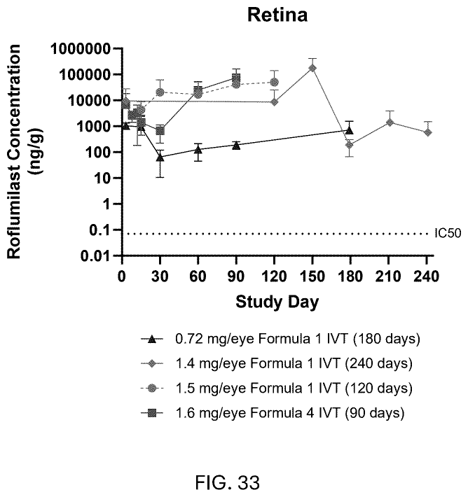

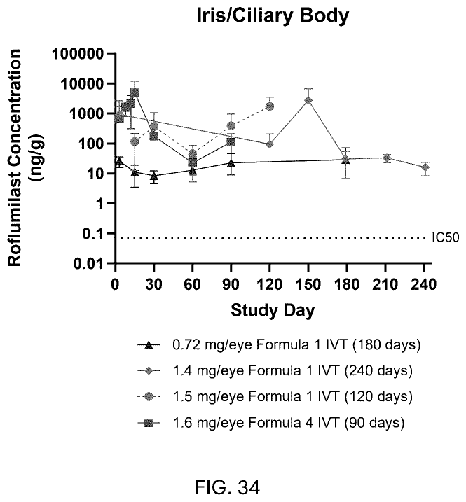

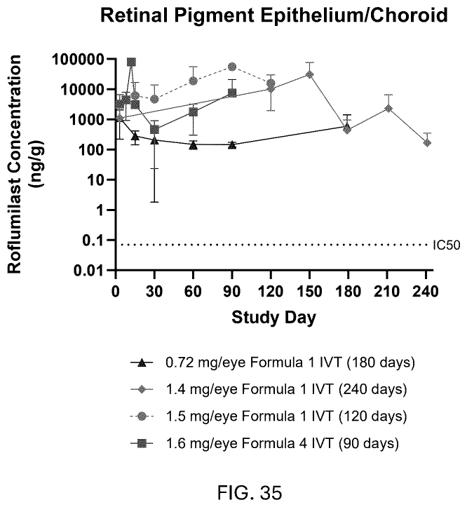

FIG. 30 through 35 are composite graphs of four separate rabbit model studies showing the mean #standard error of the mean (SEM) tissue residence of roflumilast in the aqueous humor, the VH supernatant fraction, the VH concentrate fraction, the retina, the iris/ciliary body, and the retinal pigment epithelium/choroid of the subjects, respectively. The figure legends indicate the duration of each study group. Except for FIG. 30 (aqueous humor) which reports results for three study groups, four study groups were administered with a formulation of roflumilast intravitreally (IVT). The first study group was treated with a single dose of 1.5 mg roflumilast per eye using Formulation 1 from Table 2. The second study group was treated with a single dose of 1.4 mg roflumilast per eye using Formulation 1 form Table 2. The third study group was treated with a single dose of 1.6 mg roflumilast per eye using Formulation 4 from Table 2. The fourth study group was treated with a single dose of 0.72 mg roflumilast (i.e., 25 μL instead of the 50 μL of the formulation from the second study group) per eye using Formulation 1 from Table 2. The four studies revealed that up to 240 days, the two different formulations, both designed to produce depots with increased viscosity, maintained stable tissue residence in the vitreal humor supernatant, vitreal humor concentrate, and retina at therapeutically relevant concentrations. As illustrated in FIG. 34, the four studies revealed that up to 240 days, the two different formulations were able to maintain stable residence in the iris/ciliary body at therapeutically relevant concentrations. The data reflects that the formulations are capable of maintaining therapeutic levels in the iris/ciliary body, which is a good target for certain indications, such as uveitis. As illustrated in FIG. 35, the four studies revealed that up to 240 days, the two different formulations were able to maintain stable residence in the retinal pigment epithelium/choroid. The data reflects that the formulations are capable of maintaining therapeutic levels in the retinal pigment epithelium/choroid, which is a good target for certain indications, such as retinal indications and back of the eye indications.

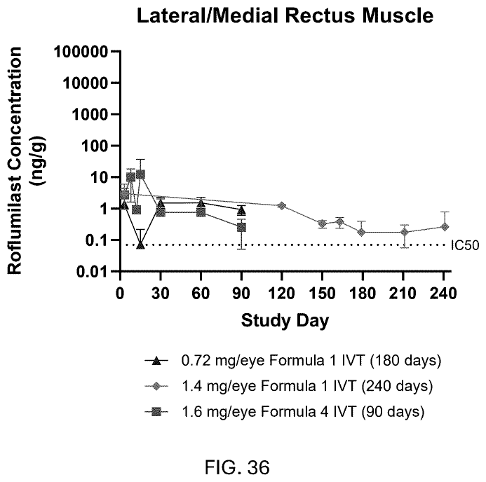

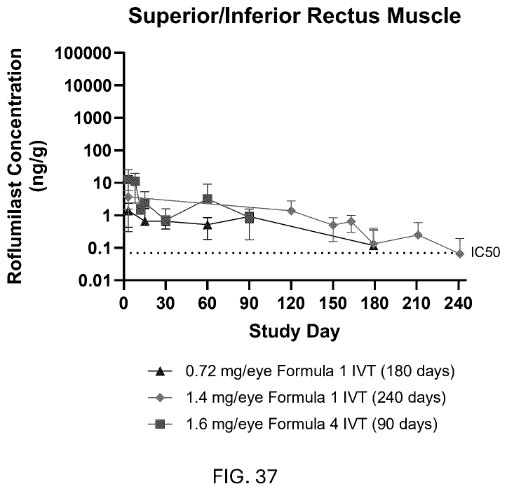

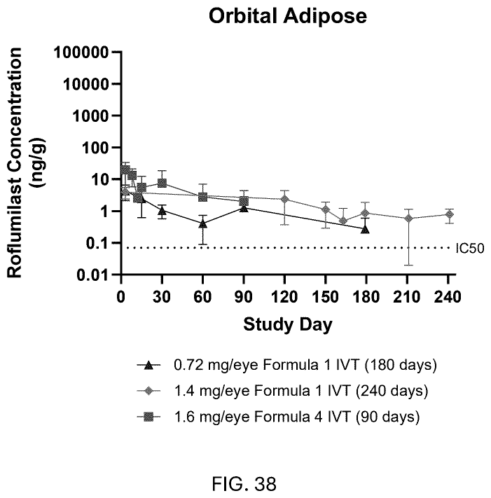

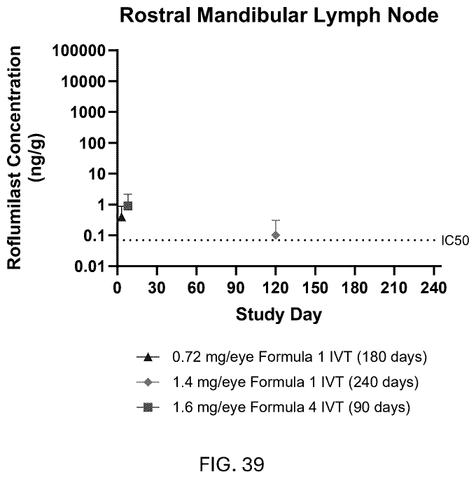

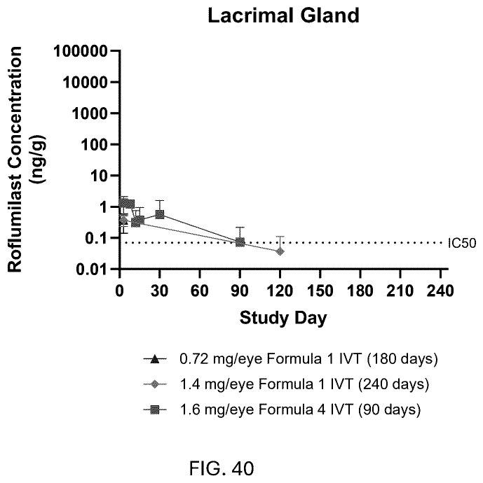

FIG. 36 through 40 are composite graphs of three separate rabbit model studies showing the mean±standard error of the mean (SEM) tissue residence of roflumilast in the lateral/medial rectus muscle, superior/inferior rectus muscle, orbital adipose, rostral mandibular lymph node, and lacrimal gland of the subjects, respectively. The figure legends indicate the duration of each study group. All three study groups were administered with a formulation of roflumilast intravitreally (IVT). The first study group was treated with a single dose of 1.4 mg roflumilast per eye using Formulation 1 form Table 2. The second study group was treated with a single dose of 1.6 mg roflumilast per eye using Formulation 4 from Table 2. The third study group was treated with a single dose of 0.72 mg roflumilast (i.e., 25 μL instead of the 50 μL of the formulation from the first study group) per eye using Formulation 1 from Table 2. The four studies revealed that in certain cases, the two different formulations maintained stable tissue residence in the lateral/medial rectus muscle, superior/inferior rectus muscle, orbital adipose, rostral mandibular lymph node, and lacrimal gland in some cases at therapeutically relevant concentrations in some cases as long as 240 days. The data reflect that the formulations are capable of maintaining therapeutic levels in these targets, which support treatment for thyroid eye disease (TED).

Interestingly, tissue residence was approximately 100-fold greater than the IC50 in most of these measured tissues. Even under these markedly higher concentrations of roflumilast, the half-dose concentrations revealed dose-dependent behavior, evidencing a pharmacodynamic profile inconsistent with simple saturation kinetics. Therefore, the tissue residence illustrates a significant fold coverage of the IC50 of key tissues for anterior, posterior, and periorbital tissues.

At the end of the study, the tolerability of roflumilast administered peribulbarly (Formulation 1 from Table 2) and intravitreally (IVT, Formulation 4 from Table 2) was assessed. IVT administration was generally well tolerated, and the formulation formed a depot with minimal strands, indicating an absence of unwanted particulate matter, fibrils, or aggregation in the depot which can lead to inflammation, toxicity, or loss of vision. Indeed, no anterior inflammation was observed in any test animal. Additionally, there were no snow globe effects observed, and the depots appeared to decrease in size over time, indicating a robust yet ultimately transient mode of drug delivery to the therapeutic target tissues of the eye. Peribulbar injections of Formulation 1 from Table 2 was also well tolerated with no observed ocular events or any other parameter.

Surprisingly, the formulations disclosed herein were of low enough viscosity to be comfortable, convenient, and safe since they do not require a significant amount of force during injection. And despite this low degree of viscosity, the formulations formed self-aggregating depots in known high viscosity systems like rabbit vitreous humor as well as low to very low viscous challenge conditions in vitro, which is important in elderly human patients who often have lower viscosity vitreous. Additionally surprising is the self-aggregation of depots without the addition of thermogels or other foreign particles which can cause inflammation. The foregoing description has been presented for purposes of illustration and description. This description is not intended to limit the invention to the precise form disclosed. Persons of ordinary skill in the art will appreciate that modifications and substitutions of the basic inventive description may be made.

1-21. (canceled)

22. A method for treating an eye disorder in a patient comprising:
injecting a high viscosity ophthalmic pharmaceutical composition of roflumilast without any foreign device or implant, into the eye of the patient,
wherein the pharmaceutical composition comprises about 1.0% to about 5% w/v of roflumilast; a viscosity agent; a tonicity agent; a buffer agent; a surfactant; and water;
and wherein the viscosity of the composition is between about 2 and about 100 cPs.

23. The method of claim 22, wherein the pharmaceutical composition provides a sustained release of roflumilast in the posterior or peri-orbital space over a period of about 30 days.

24. The method of claim 23, wherein the pharmaceutical composition remains well-tolerated throughout the duration of action.

25. The method of claim 22, wherein the pharmaceutical composition provides a sustained release of roflumilast in the posterior or periorbital space over a period of about 60 days.

26. The method of claim 25, wherein the pharmaceutical composition remains well-tolerated throughout the duration of action.

27. The method of claim 22, wherein the pharmaceutical composition provides a sustained release of roflumilast in the posterior or periorbital space over a period of about 90 days.

28. The method of claim 27, wherein the pharmaceutical composition remains well-tolerated throughout the duration of action.

29. The method of claim 22, wherein the pharmaceutical composition provides a sustained release of roflumilast over a period of about 120 days.

30. The method of claim 22, wherein the composition produces a drug depot that remains in the vitreous for up to about 30 days.

31. The method of claim 22, wherein the composition produces a drug depot that remains in the vitreous for up to about 60 days.

32. The method of claim 22, wherein the composition produces a drug depot that remains in the vitreous for up to about 90 days.

33. The method of claim 22, wherein the composition produces a drug depot that remains in the vitreous for up to about 120 days.

34. The method of claim 22, wherein the viscosity agent is selected from a group consisting of hydroxypropyl methylcellulose, polyvinylpyrrolidone, and sodium carboxymethyl cellulose.

35. The method of claim 22, wherein the tonicity agent is sodium chloride.

36. The method of claim 22, wherein the buffer is sodium acetate and sodium citrate.

37. The method of claim 22, wherein the surfactant is a polysorbate.

38. The method of claim 22, wherein the pH of the composition is between 5.5 and 7.5.

39. The method of claim 22, wherein the ophthalmic pharmaceutical formulation does not include any preservatives or anti-microbial agents.

40. The method of claim 22, wherein the pharmaceutical composition has a particle size distribution characterized by a d90 value of less than or equal to about 15 μm.

41. The method of claim 22, wherein the pharmaceutical composition has been subjected to terminal sterilization by gamma irradiation or dry heat sterilization to achieve less than about 0.2% impurities.

42. The method of claim 22, wherein the eye disorder is selected from the group consisting of anterior, posterior, pan or intermediate uveitis, uveitis associated with HLA-B27, juvenile idiopathic arthritis, Behcets disease, ankylosing spondylitis, Vogt-Koyanagi-Harada disease (VKH), or autoimmune disease, ocular graft vs host disease, Stevens-Johnson syndrome/toxic epidermal necrolysis, diabetic retinopathy, diabetic macular edema, retinal vein occlusion, age-related macular degeneration (AMD) including dry, geographic atrophy, or exudative AMD, choroidal neovascularization, retinal vasculitis (drug related/iatrogenic, non-infectious/sterile, or idiopathic), macular telangiectasia (including type 1, 2, and 3), normotensive or elevated intraocular pressure related glaucoma, thyroid eye disease (TED), choroidal thickening associated with thyroid eye disease (TED), Coats' disease, central serous retinopathy or chorioretinopathy, sterile or infectious endopthalmitis, retinitis, choroiditis, anterior or posterior sclertis/episcleritis, endothelial keratitis (bacterial, viral, fungal, or non-infectious in nature), and other inflammatory diseases of the anterior and posterior tissues of the eye or ocular complications of other inflammatory or autoimmune diseases, inflammation associated with inherited retinal diseases, retinitis pigmentosa, Stargardt disease, Leber congenital amaurosis, Leber hereditary optic neuropathy, Usher syndrome, X-linked retinoschisis, choroidemia, zonal occult outer retinopathy, myopia, vitreomacular adhesion, retinal detachment, choroidal detachment and hemorrhage, choroidal rupture, choroidal folds, proliferative vitreoretinopathy, idiopathic ischemia, achromatopsia, retinopathy of prematurity, gyrate atrophy, central areolar choroidal dystrophy, punctate inner choroidopathy, multifocal choroiditis, choroiditis, choroidal granuloma, choroidal dystrophy, choroidal fibrosis, acute posterior multifocal placoid pigment epitheliopathy, serpiginous choroidopathy, birdshot retinochoroidopathy, multiple evanescent white dot syndrome, retinoblastoma, choroidal melanoma, retinal lymphoma, or iatrogenic posterior or vitreous chamber inflammation.

43. The method of claim 22, wherein the patient remains well-tolerated during the duration of action.

44. The method of claim 22, wherein the injection is an intravitreal, sub conjunctival, sub retinal, intracameral, sub-tenon, periocular, peribulbar, retrobulbar, retro-orbital, or suprachoroidal injection.

45. The method of claim 22, wherein the pharmaceutical composition delivers a therapeutic level of roflumilast to one or more of the cornea, limbus, conjunctiva, eyelids, lacrimal and Meibomian glands, lens, pupil, iris, anterior sclera, ciliary body, lacrimal glands, aqueous humor, the internal or endothelial or inner layer of the cornea, lacrimal glands, lymph nodes, posterior sclera, retina, choroid, macula, fovea, optic disc, optic nerve, vitreous humor, hyaloid canal, or extraorbital or periorbital tissues and muscle/connective tissues.

46. The method of claim 45, wherein the pharmaceutical composition is capable of delivering therapeutically effective levels of roflumilast to one or more of the cornea, limbus, conjunctiva, eyelids, lacrimal and Meibomian glands, lens, pupil, iris, anterior sclera, ciliary body, lacrimal glands, aqueous humor, the internal or endothelial or inner layer of the cornea, lacrimal glands, lymph nodes, posterior sclera, retina, choroid, macula, fovea, optic disc, optic nerve, vitreous humor, hyaloid canal, or extraorbital or periorbital tissues and muscle/connective tissues for 240 days following administration.

47. The method of claim 46, wherein the pharmaceutical composition is capable of delivering therapeutically effective levels of roflumilast to the extraorbital or periorbital tissues for 240 days following administration.

48. The method of claim 22, wherein the viscosity of the composition is between about 2 and about 20 cPs.

49. The method of claim 22, wherein the viscosity of the composition is between about 7 and about 15 cPs.

50-82. (canceled)
